(19) United States
(12) Patent Application Publication
(10) Pub. No.: US 2023/0212340 A1
(43) Pub. Date: Jul. 6, 2023

(54) CONTRAST AGENT FOR 3D EX VIVO IMAGING OF VASCULAR AND TUBULAR STRUCTURES IN THE KIDNEY

(71) Applicant: UNIVERSITÄT ZÜRICH, Zürich (CH)

(72) Inventors: Bernhard SPINGLER, Basel (CH); Ngoc An LE, Zürich (CH); Vartan KURTCUOGLU, Winterthur (CH); Willy KUO, Regensdorf (CH)

(73) Assignee: UNIVERSITÄT ZÜRICH, Zürich (CH)

(21) Appl. No.: 17/781,731

(22) PCT Filed: Dec. 4, 2020
(86) PCT No.: PCT/EP2020/084771

§ 371 Date: Jun. 2, 2022

(30) Foreign Application Priority Data
Dec. 4, 2019 (EP) 19213688.5
May 22, 2020 (EP) 20176162.4

(51) Int. Cl. C08F 220/56; C07C 233/55

(52) U.S. Cl. CPC C08F 220/56; C07C 233/55

(57) ABSTRACT

The present invention relates to a contrast agent suitable for ex vivo imaging, particularly of vascular structures and renal tubular structures, and a method for ex vivo imaging. The contrast agent is a polymer comprising monomers M. The monomer comprises a backbone having 2 to 6 elements, wherein at least one element is —CH(R)— or —N(R)—. R is a moiety -E-H, -L-(NH2)m or a moiety of formula 1, with E, L, R¹ and R² being defined as described in the present specification. The monomer comprises at least one —I to allow detection via X-ray and at least one —NH₂ to allow crosslinking. Furthermore, the monomer comprises polar functional groups that contribute to water solubility. To avoid extravasation and glomerular filtration, the polymers are pre-crosslinked before the vasculature of a tissue, an organ or whole animal is perfused. After perfusion, the pre-crosslinked contrast agent is further crosslinked to be retained within the tissue, organ or animal permanently.

### CROSS-REFERENCE TO RELATED APPLICATIONS

This is the U.S. National Stage of International Patent Application No. PCT/EP2020/084771 filed on Dec. 4, 2020, which in turn claims the benefit of European Patent Application No. 20176162.4 filed on May 22, 2020, and European Patent Application No. 19213688.5 filed on Dec. 4, 2019.

### FIELD OF THE INVENTION

The present invention relates to a contrast agent suitable for ex vivo imaging of vascular structures e.g. in whole mouse and brain, and vascular and tubular structures e.g. in kidney. The contrast agent is detectable by imaging methods using X-ray such as X-ray microcomputed tomography (micro-CT).

### BACKGROUND OF THE INVENTION

Qualitative and quantitative assessment of vascular physiology, pathology and angiogenesis, such as in various types of cancer, myocardial infarction, stroke, atherosclerosis, vasculitis, and inflammation requires accurate three-dimensional (3D) structural data. Three-dimensional (3D) imaging provides morphometric parameters including vessel volume, connectivity, number, thickness, thickness distribution, separation, and degree of anisotropy. These parameters are not only useful for investigating diseases that affect the vasculature, but are also crucial for proper evaluation of pro- and anti-angiogenic therapies in preclinical models. 2D imaging techniques only provide some of these measures, and are limited to sampling sub-volumes and extrapolation to the whole organ scale.

For example, structural imaging of kidney vasculature can be used to investigate a wide variety of kidney functions. For example, it was used in the evaluation of capillary rarefaction in ischemia-reperfusion, unilateral ureteral obstruction and Alport models of kidney disease, measurement of vascular volume in different kidney regions, changes of kidney cortical vascular volume in chronic bile duct ligation model of liver cirrhosis, measurement of vessel area of wrapped artery-vein pairs permitting oxygen shunting, and analysis of the blood vessel hierarchy and bifurcations.

There are a number of methods available to investigate the vascular structure, namely histology, serial sectioning, tissue clearing and X-ray microcomputed tomography (micro-CT) on vascular casts.

In histology, structures are evaluated on a representative number of samples of two-dimensional tissue sections and statistically extrapolated to the whole volume of the respective organ. This approach is not only labor-intensive, but there are also a variety of potential traps if correct stereological procedures are not strictly adhered to. The reference trap, for example, refers to erroneous extrapolation of the evaluated features if the reference volume is incorrectly determined, e.g. if sample shrinkage during paraffin embedding is not accounted for. Or insufficiently isotropic uniform random sampling may lead to false estimation of features with preferential direction, such as the highly parallel vasa recta in the inner medulla.

Whole organ imaging, on the other hand, not only provides unbiased isotropic sampling of the full organ, but also provides non-statistical information such as the three-dimensional (3D) structural arrangement of blood vessels. These data can furthermore be used to obtain otherwise inaccessible information such as diffusion distance maps or connectivity analyses, and can be used to calculate transport and distribution of compounds such as oxygen. Such modeling is dependent on accurate 3D structural information of both the blood vessels, the main oxygen suppliers, and the main oxygen consumers. In the kidney, tubules are the main oxygen consumers.

In vivo vascular 3D imaging techniques like magnetic resonance imaging (MRI), positron emission tomography (PET) and clinical X-ray computed tomography (CT) do not provide sufficient spatial resolution to visualize capillaries, which have diameters of around 4 to 10 μm. In addition to the technical limitations of these imaging methods, resolution is further limited in vivo by movement during respiratory and cardiac cycles. These methods are, therefore, not suitable for imaging at capillary scale.

In contrast, ex vivo high-resolution X-ray imaging is not limited by anaesthetic tolerance or the dose of ionizing radiation. In addition, organs can be extracted and imaged at smaller field of views, resulting in a corresponding increase of resolution in cone-beam μCT.

The density difference between blood and soft tissue is, however, too small to be captured with standard absorption contrast using laboratory sources. Radiopaque X-ray contrast agents featuring heavy atomic elements have to be injected into the vasculature to provide the necessary contrast.

Serial sectioning extends the histological method to the third dimension, but still suffers from various sample preparation artifacts, notably shrinkage during dehydration and cutting artifacts during sectioning. These artifacts complicate realignment and virtual reassembly of the sections, requiring non-rigid registration algorithms if structural data need to be preserved.

In recent years, a large variety of tissue clearing methods have been introduced. These allow lightsheet microscopy on intact, uncut tissue by reducing light scattering via a variety of methods such as refractive index matching or lipid removal. However, residual light scattering reduces the achievable resolution at depth and leads to optical distortion, and the clearing process typically introduces swelling or shrinkage of the sample of 20% by length. Tissue clearing with lightsheet microscopy is therefore well suited for large, well separated features at the surface of the kidney, such as glomeruli, but not for capturing the dense capillary network in the depth of the kidney.

Compared to visible light, X-rays penetrate soft tissue with negligible absorption or refraction, which allows X-ray microcomputed tomography (micro-CT) to provide 3D data with isotropic quality and resolution regardless of depth within the sample. Organs can be imaged fully intact in their native wet state, preventing any additional sample distortion after fixation through swelling, shrinkage or cutting. The geometric magnification used in cone-beam μCT allows for continuously variable pixel sizes and for hierarchical imaging on low-resolution animal scale and high-resolution organ scale in a single device. Whole small animals and organs can be imaged in their entirety in their native hydrated state, minimizing sample distortion through sample preparation artefacts, dehydration, realignment artefacts or optical distortion.

TABLE 1
Current kidney vascular imaging methods and their limitations

| Method | Limitations |
|---|---|
| Sections with | Cutting artifacts |
| stereological | Shrinkage |
| quantification | No 3D structural data |
| Serial sections | Cutting artifacts |
| | Shrinkage |
| | Realignment artifacts |
| Tissue clearing | Swelling or shrinkage |
| | Optical distortion |
| X-ray micro-CT | Requires contrast agent |
| | Vascular imaging only |

To get sufficient absorption in blood vessels for contrast, X-ray contrast agents have to be injected into the vasculature. While this ensures that only functional, actively perfused blood vessels are measured, contrast agent filling has to be reliable and representative of the vascular network. Standard clinical angiography contrast agents such as iopamidol and iohexol are not suitable for vascular imaging, as they are low molecular weight compounds capable of passing the blood vessel walls quickly, leading to a loss of contrast between the vascular lumen and the surrounding tissue within a few minutes. They are furthermore cleared via renal glomerular filtration, which is necessary to prevent their accumulation in the patient's body. However, this reduces their suitability for renal imagine, since contrast is introduced into the renal tubular lumen as well, preventing clear separation of vascular and tubular structures

Blood pool contrast agents are designed for longer circulation times on the scale of hours and surface-functionalized metal nanoparticles are available in sizes that avoid glomerular filtration. However, their tendency in ex vivo settings to sediment, aggregate and diffuse out of the vasculature makes them unsuitable for capillary imaging in the kidney (FIG. 9, see also Kuo et al. 2019).

Plastic resins such as Microfil (Microfil, Flow Tech, Carver, Mass.), PU4ii (vasQtec, Zurich, Switzerland) and pAngiofil (Fumedica AG, Muri, Switzerland) that are used in vascular casting are hydrophobic and do not diffuse through the hydrated endothelial cells. They polymerize after injection and are thus retained permanently in the vasculature. However, due to their hydrophobicity, any water-based fluid present in the vasculature must be displaced physically during the injection. If water is incompletely removed, water inclusions can result in visible bubbles instead of fully filled large vessels (Ehling et al., 2016 and Vasquez et al. 2011). To reduce this problem, flow rate and thus perfusion pressure is typically increased, which may lead to overinflation of the vessels. In the kidney, the increased perfusion pressure may lead to bleeding into the renal capsule, visible as shape distortion of the kidney surface. The renal tubular lumen may also collapse due to the lack of tubular counter pressure in the absence of glomerular filtration during perfusion of a hydrophobic substance (Hlushchuk et al., 2018). Beyond those potential artifacts, current X-ray micro-CT can only capture the contrast agent (rather than the native tissue), which is solely present in the vasculature in this approach. Unlike the other methods, it can therefore not provide structural data of the renal tubular tissue directly.

Well-optimized injection techniques, closure of blood vessels via ligation in order to divert all flow to the organ of interest and high perfusion pressures are, therefore, required to obtain consistent perfusion results. While some of these problems can be attributed to the high viscosity of the polymerizing plastic resins, they are inherent limitations shared by hydrophobic casting materials. Even low viscosity gaseous carbon dioxide is unable to fill capillaries below 8 μm in diameter. While plastic resins can provide reasonable vascular filling, extensive practice is required to reliably prevent frequent sample preparation failures and incomplete vessel filling. Reliable vessel filling is, however, absolutely required for the quantitative characterization of pathological processes and the comparison of vascular phenotypes, as the resulting structural data is otherwise dominated by sample preparation artefacts and not representative of the true vascular structure.

There is thus still a need for an imaging protocol that provides artifact- and distortion-free 3D vascular imaging, and in particular for concurrent vascular and tubular 3D imaging in whole kidneys. As only X-ray micro-CT of the abovementioned methods is capable of distortion-free imaging, the shortcomings of the previous X-ray contrast agents were addressed. To that end, the inventors developed a novel iodine-based X-ray contrast agent specifically for ex vivo X-ray micro-CT. It is a water-soluble polymer large enough to avoid glomerular filtration and can be cross-linked with glutaraldehyde to be retained within the vasculature permanently. It combines the reliable, low-resistance filling of hydrophilic contrast agents with the permanent retention of vascular casting resins. This not only permits extraction of the organs without leakage of the contrast agent, but also enables the longer scan time required for high-resolution ex vivo imaging or multiple scans of the same sample, such as for hierarchical imaging or dual-energy micro-CT applications.

Here the inventors report on a kidney-specific protocol with this new contrast agent, which does not only provide vascular 3D imaging, but tubular imaging as well. The inventors further provide an image processing and quantification workflow that requires no specialized image processing expertise and relies solely on the freely available Fiji/ImageJ software platform.

Based on the above-mentioned state of the art, the objective of the present invention is to provide means and methods to image vascular and tubular structures ex vivo by using a water-soluble, aldehyde fixable and long-term stable contrast agent. This objective is attained by the subject-matter of the independent claims of the present specification.

### DESCRIPTION OF THE INVENTION

### Terms and Definitions

Unless defined otherwise, all technical and scientific terms used herein have the same meaning as commonly understood by one of ordinary skill in the art (e.g., in cell culture, biochemistry, histology, radiology). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term RAFT in the context of the present specification relates to Reversible Addition-Fragmentation chain Transfer (RAFT).

The term RAFT agent in the context of the present specification relates to an initiator of polymerization in Reversible Addition-Fragmentation chain Transfer (RAFT) reaction. For example, 2-(Dodecylthiocarbonothioylthio)-2-methylpropionic acid (CAS 461642-78-4) is a suitable RAFT agent.

In the context of the present invention, imaging of vascular structure or imaging of blood vessels relates to the visualization of the inner volume of blood vessels that is filled with the contrast agent according to the invention. For this, the vasculature is perfused using the contrast agent according to the invention, particularly the pre-crosslinked polymer. After further crosslinking, the contrast agent, particularly the crosslinked polymer, is detected using X-ray.

Imaging of renal tubular structures or imaging of renal tubules relates to the visualization of tubular cells that comprise the contrast agent according to the invention. The staining is achieved by perfusing the vasculature using the contrast agent as described above and can be detected by using X-ray.

### DESCRIPTION

The present invention aims to provide means and methods to image ex vivo the inner structure of organs, particularly vascular and renal tubular structures. This objective is attained by a contrast agent as described in the first to fourth aspect of the invention and an imaging method as described in the fifth aspect of the invention.

Imaging of vascular and renal tubular structures, e.g. of the kidney of a mouse, is performed in several steps. Depending on the experimental step, the contrast agent may be present as polymer, as pre-crosslinked polymer or crosslinked polymer. The polymer comprises several monomers M.

A first aspect of the invention relates to a monomer M or a salt thereof. The monomer comprises a backbone with 2 to 6 elements B, wherein
each B independently from any other B is selected from —CH₂—, —NH—, —C(═O)—, —CH(R)— and —NR—, wherein
   B may optionally be substituted by a C₁₋₄-alkyl, in particular by ethyl or methyl, more particularly by methyl, and wherein
   at least one of the moieties —CH(R)— and —NR— is present in the backbone,
R is independently selected from -E-H, -L-(NH₂)ₘ, and a moiety of formula 1,

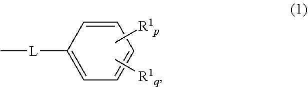

wherein
   E is a moiety comprising one or more moieties, particularly 1 to 3 moieties, independently selected from —C(═O)—, —NH—C(═O)—, —O—, —C₁₋₄-alkyl-,
   L is a linker comprising one or more moieties, particularly 1 to 3 moieties, independently selected from —C(═O)—, —C(═O)—NH—, —NH—C(═O)—, —O—, —C₁₋₄-alkyl-,
      wherein L may optionally be substituted by -E-H,
   R¹ is —I,
   R² is -E-H or -L-(NH₂)ₘ,
      p is independently selected from 0, 1, 2 or 3
      q is independently selected from 0, 1, 2, 3 or 4, particularly 0, 1 or 2,
      wherein the sum of p and q in formula 1 is ≤5,
      m is independently selected from 1 or 2, wherein
the sum of all m in the monomer is ≥1, particularly ≥2, and
the sum of all p in the monomer is ≥1, particularly the sum is 2 or 3, more particularly the sum is 3.

A monomer according to the first aspect of the invention may comprise 2 to 6 elements B, which form a backbone. Some elements B may function as a spacer, e.g. —CH₂—, —NH—, —C(═O)—, whereas other elements B comprise a residue R (—CH(R)— and —NR—).

The residue R may comprise linear (-E-H, -L-(NH₂)ₘ) or cyclic scaffold components (moiety of formula 1), which comprise functional groups such as —C(═O)—, —C(═O)—NH—, —NH—C(═O)—, —O— that contribute to the water solubility of the contrast agent. Water soluble contrast agents allow a reliable, low-resistance filling without the formation of water inclusions. In contrast to this, there is a risk of water inclusions that result in visible bubbles when using a hydrophobic contrast agent such as plastic resins. To reduce this risk, the flow rate and thus perfusion pressure is typically increased for hydrophobic contrast agents, which may lead to bleeding of plastic resins, distension of the blood vessels and compression of surrounding tissues. In the kidneys, this compression may lead to the collapse of the tubular lumen.

Highly water-soluble X-ray contrast agents according to the invention inherently avoid issues with water inclusions and high flow resistance of the hydrophobic vascular casting resins, as well as the sedimentation and aggregation problems of nanoparticle suspensions.

To allow the detection via X-ray, at least one residue R comprises at least one —I. Thus, the sum of all p in the monomer is ≥1. The contrast of the image is better the more substituents —I are present in a monomer.

In certain embodiments, the sum of all p in the monomer is 2 or 3.

In certain embodiments, the sum of all p in the monomer is 3.

High molecular weight contrast agents, e.g. with a molecular weight above 65 kDa, cannot pass through blood vessel walls. The monomers according to the invention form a polymer, for example by RAFT polymerization. Typically the polymer has a molecular weight of more than 30 000 Da. To obtain high molecular weight contrast agents with a molecular weight above 65 kDa, the polymers are crosslinked. To allow crosslinking, the monomer comprises at least one free amine, i.e. the sum of all m in the monomer is ≥1.

In certain embodiments, the sum of all m in the monomer is ≥2.

In certain embodiments, the sum of all m in the monomer is between 1 and 6.

In certain embodiments, the sum of all m in the monomer is between 1 and 4.

In certain embodiments, the sum of all m in the monomer is between 2 and 4.

Before crosslinking, the free amine may be activated by forming a salt, e.g. an HCl addition salt (—NH₃⁺Cl⁻).

In certain embodiments, each B independently from any other B is selected from —CH₂—, —NH—, —C(═O)— and —CH(R)—.

The backbone of the monomer may be a peptide backbone or an aliphatic backbone such as acrylamide derived backbones or methacrylamide derived backbones. A peptide backbone may be obtained by standard protein chemistry using amino acids having a free amino group such as lysine and amino acids modified with iodine such as diiodotyrosine. Acrylamide or methacrylamide derived backbones are obtained by radical polymerization such as reversible addition-fragmentation chain transfer (RAFT). The synthesis of an acrylamide derived backbone is shown e.g. in Scheme 1 (see below, synthesis of compound 3 in section “Synthesis of the contrast agent”).

—CH₂—CH(R′)—CH₂—CH(R″)—, —CH₂—CH(R′)— are examples for acrylamide derived backbones. —CH₂—C(CH₃)(R′)—CH₂—C(CH₃)(R″)— or —CH₂—C(CH₃)(R′)— are examples for methacrylamide derived backbones.

In certain embodiments, the backbone is
a peptide backbone —C(═O)—CH(R′)—NH—C(═O)—CH(R″)—NH— or —C(═O)—CH(R′)—NH—, or
an aliphatic backbone —CH₂—CH(R′)—CH₂—CH(R″)—, —CH₂—CH(R′)—, —CH₂—C(CH₃)(R′)—CH₂—C(CH₃)(R″)— or —CH₂—C(CH₃)(R′)—, wherein

R′ and R″ consist of moieties that are selected from moieties as defined for R, wherein R′ and R″ differ from each other, particularly one of R′ and R″ is a moiety of formula 1 and the other one is -E-H or -L-(NH₂)ₘ, more particularly R′ is a moiety of formula 1 and R″ is -E-H or -L-(NH₂)ₘ.

Within one backbone, the specific moiety selected for R′, e.g. lysine, differs from the specific moiety selected for R″, e.g. diiodotyrosine.

In certain embodiments, the backbone is
a peptide backbone —C(═O)—CH(R′)—NH—C(═O)—CH(R″)—NH— or —C(═O)—CH(R′)—NH—, or
an aliphatic backbone —CH₂—CH(R′)—CH₂—CH(R″)— or —CH₂—CH(R′)—.

In certain embodiments, the backbone is
a peptide backbone —C(═O)—CH(R′)—NH—C(═O)—CH(R″)—NH— or —C(═O)—CH(R′)—NH—, or
an aliphatic backbone —CH₂—CH(R′)—.

In certain embodiments, the backbone is an aliphatic backbone —CH₂—CH(R′)—CH₂—CH(R″)—, —CH₂—CH(R′)—, —CH₂—C(CH₃)(R′)—CH₂—C(CH₃)(R″)— or —CH₂—C(CH₃)(R′)— with R′ and R″ as defined above.

In certain embodiments, the backbone is an aliphatic backbone —CH₂—CH(R′)—CH₂—CH(R″)— or —CH₂—CH(R′)—, particularly —CH₂—CH(R′)— with R′ and R″ as defined above.

To enhance the water solubility of the monomer, it may comprise hydrophilic moieties such as —OH and —COOH.

In certain embodiments, -E-H is independently selected from —OH, —C₁₋₄-alkyl-OH, —C(═O)— OH, —C₁₋₄-alkyl-C(═O)—OH, —O—C₁₋₄-alkyl and —C₁₋₄-alkyl-O—C₁₋₄-alkyl.

In certain embodiments, -E-H is independently selected from —OH and —C(═O)—OH.

In certain embodiments, R is independently selected from -L-(NH₂)ₘ and a moiety of formula 1.

In certain embodiments, -L-(NH₂)ₘ is independently selected from —C₁₋₄-alkyl-NH₂, —C₁₋₄-alkyl-C(═O)—NH₂, —C(═O)—NH₂, —C(═O)—NH—C₁₋₄-alkyl-NH₂, —NH—C(═O)—C₁₋₄-alkyl-NH₂ and —O—C₁₋₄-alkyl-NH₂ in case of R being -L-(NH₂)ₘ.

To enhance the water solubility, the alkyl moieties are short.

In certain embodiments, -L-(NH₂)ₘ is independently selected from —C₁₋₂-alkyl-NH₂, —C₁₋₂-alkyl-C(═O)—NH₂, —C(═O)—NH₂, —C(═O)—NH—C₁₋₂-alkyl-NH₂, —NH—C(═O)—C₁₋₂-alkyl-NH₂ and —O—C₁₋₂-alkyl-NH₂ in case of R being -L-(NH₂)ₘ.

In certain embodiments, -L-(NH₂)ₘ is independently selected from —C(═O)—NH₂ or —C₁₋₄-alkyl-NH₂ in case of R being -L-(NH₂)ₘ.

In certain embodiments, -L-(NH₂)ₘ is —C(═O)—NH₂ in case of R being -L-(NH₂)ₘ.

In certain embodiments, -L-(NH₂)ₘ is —C₁₋₄-alkyl-NH₂ in case of R being -L-(NH₂)ₘ. For example, -L-(NH₂)ₘ is —C₁₋₄-alkyl-NH₂ such as in the side chain of lysine.

In certain embodiments, -L-(NH₂)ₘ is independently selected from —C₁₋₄-alkyl-NH₂, —C₁₋₄-alkyl-C(═O)—NH₂, —C(═O)—NH₂, —C(═O)—NH—C₁₋₄-alkyl-NH₂, —NH—C(═O)—C₁₋₄-alkyl-NH₂ and —O—C₁₋₄-alkyl-NH₂ in case of R² being -L-(NH₂)ₘ.

To enhance the water solubility, the alkyl moieties are short.

In certain embodiments, -L-(NH₂)ₘ is independently selected from —C₁₋₂-alkyl-NH₂, —C₁₋₂-alkyl-C(═O)—NH₂, —C(═O)—NH₂, —C(═O)—NH—C₁₋₂-alkyl-NH₂, —NH—C(═O)—C₁₋₂-alkyl-NH₂ and —O—C₁₋₂-alkyl-NH₂ in case of R² being -L-(NH₂)ₘ.

In certain embodiments, -L-(NH₂)ₘ is independently selected from —C(═O)—NH—C₁₋₂-alkyl-NH₂ in case of R² being -L-(NH₂)ₘ.

In case the backbone is a peptide backbone —C(═O)—CH(R′)—NH—C(═O)—CH(R″)—NH—, one of the moieties R′ and R″ may be a moiety of formula 1 and the other moiety of R′ and R″ may be -L-NH₂. In certain embodiments, one of the moieties R′ and R″, particularly R′, is a moiety of formula 1 with p being 2 or 3, particularly 2, and q being 1 or 2, particularly 1, and R² being -E-H, particularly R² being —OH, —C₁₋₄-alkyl-OH, —COOH, more particularly —OH. In certain embodiments, one of the moieties R′ and R″, particularly R″, is -L-NH₂ and L is a C₁₋₄-alkyl, particularly a C₃₋₄-alkyl. In certain embodiments, one of the moieties R′ and R″, particularly R′, is a moiety of formula 1 with p being 2 or 3, particularly 2, and q being 1 or 2, particularly 1, and R² being -E-H, particularly R² being —OH, —C₁₋₄-alkyl-OH, —COOH, more particularly —OH, and the other moiety of R′ and R″, particularly R″, is -L-NH₂ with L being a C₁₋₄-alkyl, particularly a C₃₋₄-alkyl.

In case the backbone is an aliphatic backbone —CH₂—CH(R′)—CH₂—CH(R″)—, one of the moieties R′ and R″, particularly R′ is selected from a moiety of formula 1 with p being 2 or 3, particularly 3, q being 1 or 2, particularly 2, R² being -E-H with E being —C(═O)—O— or C₁₋₄-alkyl-COO—, particularly —COO—. In certain embodiments, one of the moieties R′ and R″, particularly R″, is -L-(NH₂) with L being —C(═O)— or —C₁₋₄-alkyl-, particularly —C(═O)—. In certain embodiments, one of the moieties R′ and R″, particularly R′ is selected from a moiety of formula 1 with p being 2 or 3, particularly 3, q being 1 or 2, particularly 2, R² being -E-H with E being —C(═O)—O— or C₁₋₄-alkyl-COO—, particularly —COO—, and the other of the moieties R′ and R″, particularly R″, is -L-(NH₂) with L being —C(═O)— or —C₁₋₄-alkyl-, particularly —C(═O)—.

In case the backbone is an aliphatic backbone —CH₂—CH(R′)—, R′ is selected from a moiety of formula 1 with p being 2 or 3, particularly 3, q being 1 or 2, particularly 2, R² being -L-(NH₂) with L being —C(═O)—NH—C₁₋₄-alkyl-.

A second aspect of the invention relates to a polymer P. The polymer P comprises monomers M according to the first aspect of the invention.

The polymer P may be obtained by radical polymerization such as reversible addition-fragmentation chain transfer (see section “Synthesis of the contrast agent” and the reaction shown in Scheme 1) or by peptide synthesis.

The polymer length typically varies between 70 and 600 monomers.

In certain embodiments, the polymer P comprises 70 to 600 monomers according to the first aspect of the invention.

In certain embodiments, the polymer P comprises 100 to 300 monomers according to the first aspect of the invention.

In certain embodiments, the polymer P comprises 100 to 200 monomers according to the first aspect of the invention.

In certain embodiments, the polymer P comprises 120 to 170 monomers according to the first aspect of the invention.

In certain embodiments, the polymer P comprises in average 150 monomers according to the first aspect of the invention.

In certain embodiments, the polymer P comprises 150 monomers according to the first aspect of the invention.

The polymer P may be obtained by peptide synthesis or radical polymerization such as reversible addition-fragmentation chain transfer (RAFT). Thus, the beginning and end of the polymer are either formed by moieties derived from a radical initiator/a RAFT agent or they are an N- and C-terminus, i.e. —NH₂ and —COOH, respectively. The N- and C-terminus may be further modified, for example the C-terminus may be an amide —CONH₂.

A RAFT reaction is started by a free-radical source. For instance, the radical initiator AIBN (2,2′-Azobis(2-methylpropionitrile), 2-(azo(1-cyano-1-methylethyl))-2-methylpropane nitrile) may decompose to form a first radical RI-. Subsequently, the radical fragment may react with n monomeric educts comprising an acrylamide or methacrylamide moiety (e.g. compound 2 in Scheme 1 in section “Synthesis of the contrast agent”, intermediate M′ of formula 5) to from the propagating radical RI-[M]ₙ- (initiation and propagation).

In the presence of a RAFT agent (also referred to as chain transfer agent, CTA), the propagating radical RI-[M]ₙ- reacts with the RAFT agent to form a RAFT adduct radical RI-[M]ₙ-RAFT-. This reaction is reversible, i.e. the RAFT adduct radical may again form a propagating radical and a RAFT agent. Alternatively, the RAFT adduct radical RI-[M]ₙ-RAFT- may release a new radical FT- and form a dormant chain RI-[M]ₙ-RA. These reactions are referred to as RAFT pre-equilibrium.

The new radical FT- may give rise to a new propagating radical FT-[M]ₙ- (re-initiation).

In the main RAFT equilibrium reactions, a dormant chain, e.g. RI-[M]ₙ-RA, may react in a reversible reaction with a new propagating radical to form an intermediate radical RI-[M]ₙ-RA-[M]ₙ-FT. This intermediate radical may either release a propagating radical RI-[M]ₙ- or FT-[M]ₙ- and form the dormant chains RA-[M]ₙ-FT and RI-[M]ₙ-RA, respectively. Further intermediate radicals that may be formed include RI-[M]ₙ-RA-[M]ₙ-RI and FT-[M]ₙ-RA-[M]ₙ-FT. Depending on the availability of RAFT agents, monomeric educts, propagating radicals and dormant chains, an equilibrium will be formed between the dormant species and the propagating radicals.

Products of a RAFT reaction include the polymers RI-[M]ₙ-RA, FT-[M]ₙ-RA, RI-[M]ₙ-RI and FT-[M]ₙ-FT.

The sulfur-containing RA-adduct can be cleaved after polymerization from the polymer chain.

These polymers provide lower autofluorescence and thus higher suitability for fluorescence microscopy imaging than the RA-adduct containing embodiments.

In certain embodiments, the polymer is a compound of formula 2, 2a or 3, particularly of formula 2 and 2a, more particularly of formula 2,

X-[M]ₙ-Y (2), Z-[M]ₙ-R^{S} (2a), R^{N}-[M]ₙ-R^{C} (3), wherein

X and Y are independently from each other selected from RA, FT and RI,

Z is selected from FT and RI, wherein

RI is a moiety derived from a radical initiator, particularly from a radical initiator selected from a peroxide, a perester or an azo initiator, more particularly from AIBN, 1,1′-azobis (cyclohexanecarbonitrile), 4,4′-azobis(4-cyanopentanoic acid), 4,4′-azobis(4-cyanopentan-1-ol), 2,2′-azobis(methyl isobutyrate), 2,2′-azobis(2-cyano-2-butane), 2-(t-butylazo)-2-cyanopropane, 2,2′-azobis(N,N′-dimethyleneisobutyramine), 2,2′-azobis[2-methyl-(N)-(1,1)-bis(hydroxymethyl)-2-hydroxyethyl]propionamide, 2,2′-azobis[2-methyl-N-hydroxyethyl)]propionamide, 2,2′-azobis(2,2,4-trimethylpentane), 2,2′-azobis(2-methylpropane), t-butylperoxy isobutyrate, dibenzoyl peroxide, potassium peroxydisulfate, ammonium peroxydisulfate, di-tert-butyl peroxide, di-t-butyl hyponitrite, dicumyl hyponitrite,

RA is a RAFT (reversible addition fragmentation chain transfer) agent without the homolytic leaving group,

M is a monomer according to claim 1 or a salt thereof,

n is 70 to 600, particularly 100 to 300, more particularly 120 to 170,

FT is the homolytic leaving group of a RAFT agent or the homolytic leaving group of a RAFT agent modified by -E-H or -L-(NH₂)ₘ, wherein -E-H and -L-(NH₂)ₘ are defined as described above,

R^{S} is H or OH

R^{N} is —NH₂,

R^{C} is —COOH or —CONH₂.

In certain embodiments, the polymer is a compound of formula 2 or 3, particularly of formula 2, X-[M]ₙ-Y (2), R^{N}-[M]ₙ-R^{C} (3), wherein

X and Y are independently from each other selected from RA, FT and RI, wherein

RI is a moiety derived from a radical initiator,

RA is a RAFT (reversible addition fragmentation chain transfer) agent without the homolytic leaving group,

M is a monomer according to claim 1 or a salt thereof,

n is 70 to 600, particularly 100 to 300, more particularly 100 to 200, even more particularly 120 to 170,

FT is the homolytic leaving group of a RAFT agent or the homolytic leaving group of a RAFT agent modified by -E-H or -L-(NH₂)ₘ, wherein -E-H and -L-(NH₂)ₘ are defined as described above,

R^{N} is —NH₂,

R^{C} is —COOH or —CONH₂.

In certain embodiments, the compound of formula 2 is selected from RI-[M]ₙ-RA, FT-[M]ₙ-RA, RI-[M]ₙ-RI and FT-[M]ₙ-FT.

In certain embodiments, the polymer is a compound of formula 2a is selected from RI-[M]ₙ-R^{S} or FT-[M]ₙ-R^{S} with R^{S} being H or OH.

In certain embodiments, RI is a moiety derived from a radical initiator selected from a peroxide, a perester, an azo initiator.

In certain embodiments, RI is a moiety derived from a radical initiator selected from a AIBN, 1,1′-azobis (cyclohexanecarbonitrile), 4,4′-azobis(4-cyanopentanoic acid), 4,4′-azobis(4-cyanopentan-1-ol), 2,2′-azobis(methyl isobutyrate), 2,2′-azobis(2-cyano-2-butane), 2-(t-butylazo)-2-cyanopropane, 2,2′-azobis(N,N′-dimethyleneisobutyramine), 2,2′-azobis[2-methyl-(N)-(1,1)-bis(hydroxymethyl)-2-hydroxyethyl]propionamide, 2,2′-azobis[2-methyl-N-hydroxyethyl)]propionamide, 2,2′-azobis(2,2,4-trimethylpentane), 2,2′-azobis(2-methylpropane), t-butylperoxy isobutyrate, dibenzoyl peroxide, potassium peroxydisulfate, ammonium peroxydisulfate, di-tert-butyl peroxide, di-t-butyl hyponitrite, dicumyl hyponitrite.

In certain embodiments,
RI is a moiety of formula 5 or 6,

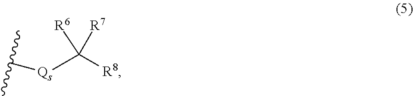

—O—SO₂—O⁻T⁺ (6), wherein
   R⁶ is selected from —C₁₋₆-alkyl, —H,
   R⁷ is selected from —C₁₋₆-alkyl, -phenyl, —C₁₋₆-alkyl-OH, —C₁₋₆-alkyl-COOH, —COOH, —C(═O)—O—C₁₋₄-alkyl, —C(═O)—NH—R⁹ with R⁹ being —C₁₋₆-alkyl-(OH)ᵣ with r being 0, 1, 2 or 3,
   R⁸ is —C₁₋₆-alkyl, —H, —CN, or
   R⁶ and R⁷ form a C₃₋₈-cycloalkyl, particularly a C₅₋₆-cycloalkyl, and R⁸ is —C₁₋₆-alkyl, —H, —CN,
   Q is —O—, —O—C(═O)— or —C(═O)—O— with s being 0 or 1,
   T⁺ is a monovalent cation, particularly Na⁺, K⁺, NH₄⁺, H⁺, selected from —C(CH₃)₂(CN) (derived from AIBN) or —O—C(═O)-phenyl (derived from dibenzoylperoxide), and/or
RA is —S—C(═S)—Z with Z being selected from phenyl and —S—C₆₋₂₀-alkyl, particularly phenyl and —S—C₁₀₋₁₆-alkyl, more particularly —S—C₁₀₋₁₆-alkyl, and/or
FT is a moiety of formula 4,

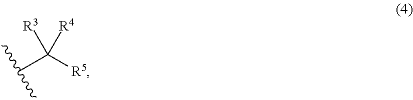

wherein
R³ is selected from —H and —C₁₋₄-alkyl, particularly —H and —C₁₋₂-alkyl,
R⁴ is selected from —H, —C₁₋₄-alkyl, —C₁₋₄-alkyl-COOH, —C₁₋₄-alkyl-C(═O)—R⁶ with R⁶ being -E-H or -L-(NH₂)ₘ, particularly —H, —C₁₋₂-alkyl, —C₁₋₂-alkyl-COOH, —C₁₋₂-alkyl-C(═O)—R⁵ with R⁵ being -E-H or -L-(NH₂)ₘ,
R⁵ is selected from —CN and —COOH, particularly R⁵ is —COOH.

In certain embodiments, RI is selected from —C(CH₃)₂(CN) (derived from AIBN) or —O—C(═O)— phenyl (derived from dibenzoylperoxide).

As the polymer itself is not large enough to avoid renal glomerular filtration, entry into the interstitial space and extravasation, it may be pre-crosslinked. Suitable functional groups for crosslinking are amine groups, particularly primary amines that are not sterically hindered. Free amine groups allow crosslinking with crosslinking agents that comprise one or more aldehyde moieties such as glutaraldehyde. By adjusting the ratio of glutaraldehyde to amines, e.g. 1:20, a pre-crosslinked polymer with a molecular weight 65 kDa is obtained. The pre-crosslinked polymer still has a low viscosity but is sufficiently large in molecular weight to avoid extravasation, entry into the interstitial space or glomerular filtration. The pre-crosslinked polymer is small enough to fill small vessels such as capillaries.

In certain embodiments formaldehyde is used for cross-linking. The use of formaldehyde provides a low autofluorescence after injection. Additionally, the effect of antigen masking of the tissue, resulting in limited capability to use the tissue for subsequent immunohistochemistry, is reduced.

A third aspect of the invention relates to a pre-crosslinked polymer. The pre-crosslinked polymer comprises two or more interconnected polymers P according to the second aspect of the invention.

In certain embodiments, the polymers are interconnected via imine bonds, which are formed by a reaction of amine moieties of the polymers and a dialdehyde or a trialdehyde, or the polymers are interconnected via a methylene bridge derived from formaldehyde.

Using a di- or trialdehyde for crosslinking, imine bonds are formed between amine moieties of the polymers the aldehyde moieties of the dialdehyde or trialdehyde.

Using formaldehyde for crosslinking, the formaldehyde reacts with an amino group of the polymer to form a Schiff base (imine). Subsequently, the imine reacts with a nucleophilic moiety of the polymer forming a methylene bridge (—CH₂—) between the N atom of the imine and the nucleophilic moiety.

In certain embodiments, the polymers are interconnected via imine bonds, which are formed by a reaction of amine moieties of the polymers and a dialdehyde or a trialdehyde.

In certain embodiments, the polymers are interconnected via imine bonds, which are formed by a reaction of amine moieties of the polymers and H—C(═O)—C₁₋₈-alkyl-C(═O)—H or benzene-1,3,5-trialdehyde, or the polymers are interconnected via a methylene bridge derived from formaldehyde.

In certain embodiments, the polymers are interconnected via imine bonds, which are formed by a reaction of amine moieties of the polymers and H—C(═O)—C₁₋₈-alkyl-C(═O)—H or benzene-1,3,5-trialdehyde.

In certain embodiments, the polymers are interconnected via imine bonds, which are formed by a reaction of amine moieties of the polymers and H—C(═O)—C₃₋₈-alkyl-C(═O)—H or benzene-1,3,5-trialdehyde, particularly H—C(═O)—C₃₋₈-alkyl-C(═O)—H.

In certain embodiments, the molecular mass of the pre-crosslinked polymer is 65 kDa.

In certain embodiments, the molecular mass of the pre-crosslinked polymer is 100 kDa.

A further aspect relates to the crosslinked polymer. The crosslinked polymer is obtained by further crosslinking the pre-crosslinked polymer. Reference is made to the embodiments described herein, particularly to the embodiments of the first to third aspect of the invention.

A fourth aspect of the invention relates to an intermediate M′. The intermediate M′ is a compound of formula 5,
D-CH(R) (5), wherein D is H₂C═ and R is a moiety of formula 1,

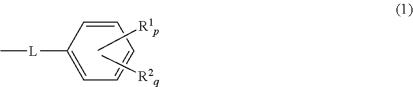

as defined above.

The double bond of the intermediate (H₂C═CH—R) may react in a radical polymerization reaction such as RAFT polymerization.

Particularly with regard to R¹ and R² of formula 1, reference is made to the embodiments of the first aspect of the invention.

In certain embodiments, the intermediate M′ is the compound M1

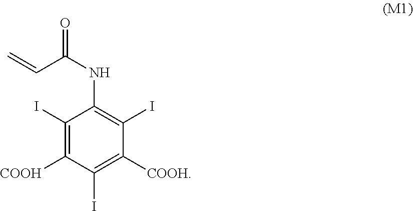 [SMILES: C=CC(=O)Nc1c(I)c(C(=O)O)c(I)c(C(=O)O)c1I]

The intermediate M′ may be synthesized as described in Scheme 1 below or according to Scheme 2, particularly as described in Scheme 1.

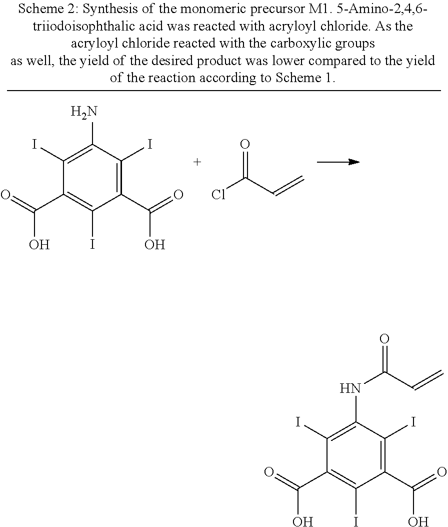

When the whole animal or an organ such as a mouse kidney is perfused with the pre-crosslinked polymer, the pre-crosslinked polymer can be further cross-linked with a crosslinking agent that comprise one or more aldehyde moieties such as glutaraldehyde to be retained within the vasculature permanently. The permanently fixed contrast agent within the blood vessel results in a stable sample that retains contrast over a long period of time.

The high-resolution contrast agent according to the invention allows not only imaging of the vasculature but also imaging of tubules in the cortex and outer medulla of a kidney. Renal glomerular filtration, entry into the interstitial space and extravasation is avoided (see FIG. 11).

A fifth aspect of the invention relates to a method for ex vivo imaging, particularly vascular imaging, more particularly vascular and renal tubular imaging. The method comprises the steps of
providing a contrast agent solution comprising the pre-crosslinked polymer according to claim 12 and a crosslinking solution comprising a crosslinking agent, particularly a crosslinking agent selected from formaldehyde a dialdehyde or a trialdehyde,
perfusing a vessel using the contrast agent solution, particularly perfusing the vasculature of a tissue, an organ or a whole animal,
adding the crosslinking solution yielding a crosslinked polymer,
detecting the crosslinked polymer using X-ray.

In certain embodiments, the vasculature of a kidney or brain is perfused.

In certain embodiments, the vasculature of a kidney is perfused. By perfusing the vasculature of a kidney, not only the inner volume can be filled with contrast agent and thus be detected.

Also renal tubular cells are stained by the contrast agent.

In certain embodiments, the crosslinking agent is selected form a dialdehyde or a trialdehyde.

In certain embodiments, the dialdehyde is selected from H—C(═O)—C₁₋₈-alkyl-C(═O)—H, particularly H—C(═O)—C₃₋₈alkyl-C(═O)—H and/or the trialdehyde is benzene-1,3,5-trialdehyde.

In certain embodiments, the tissue, organ or whole animal is immersed into the crosslinking solution.

In certain embodiments, the method comprises an image processing and quantification step.

The image processing and quantification is performed after detecting the crosslinked polymer using X-ray.

### DESCRIPTION OF THE FIGURES

FIG. 1 A: Single slice of the 3.3 μm voxel size dataset displaying large vessels, capillaries, tubular lumina and a fluid-filled structure. B: False-color image of the same slice, approximating the appearance of a histological section. Scale bars: 1 mm. C, D: Magnified views of the boxed regions in the top panels containing parts of the cortex, outer medulla and inner medulla. Scale bars: 0.5 mm.

FIG. 2 A: Overview of haematoxylin & eosin-stained histology slice. Scale bar: 1 mm. B: Part of renal cortex, containing glomeruli and an artery-vein pair. The contrast agent stains the same purple color as the tissue. Scale bar: 200 μm C: Inner stripe of the outer medulla, containing one eosinophilic protein-filled structure in the middle and vascular bundles filled with contrast agent. D: Inner medulla, containing contrast agent-filled vasa recta.

FIG. 3 Computer-rendered image of the X-ray micro-CT dataset acquired with 3.3 μm pixel size, with only tubular lumina (blue) in the top segment, vascular lumina (red) in the bottom segment and both in the middle. A: Overview image including squares indicating the origin of the regions of interest shown on the right with higher magnification. Scale bar: 1 mm. B: Tubules in the cortex. C: Tubules in the medulla. D: Fluid-filled structures in the medulla. E: Blood vessels in the medulla.

F: Blood vessels in the cortex.

FIG. 4 A: Single slice showing the diffusion distance of each voxel within the kidney to the nearest blood vessel. B: Single slice displaying the lengths of the shortest paths of each voxel to the papilla of the kidney. Scale bars: 1 mm. C: Cumulative distribution function of the diffusion distances. D: Cumulative distribution function of the blood vessel path lengths to the papilla.

FIG. 5 Synthesis pathway for compound 6, XlinCA, using RAFT polymerization.

FIG. 6 Conventional μCT images of mice heads perfused with PU4ii (A) and XlinCA (B).

In the PU4ii-filled mouse, the supraorbital vein (white arrow) is partially filled up to the bifurcation to the naso-frontal vein and anterior facial vein, which do not appear. In contrast, these vessels along with the anterior facial vein are completely filled in the XlinCA-perfused mouse. C: Maximum intensity projection of the higher resolution XlinCA-perfused whole mouse dataset. Voxel size: 20 μm, scale bar: 1 cm. D: Virtual section of the μCT dataset shown in C. Intestine (I), kidney (K), adrenal gland (AG) liver and brain are clearly visible. Scale bar: 1 cm.

FIG. 7 3D rendering of the brain hemisphere vasculature perfused with XlinCA. A: Outside view. B: Inside view. C: Magnified view of the region indicated by the black square in FIG. 4A. Diameter of blood vessel indicated by the white arrow: 70 μm. D: Magnified view of FIG. 4B. Diameter of blood vessel indicated by the white arrow: 15 μm.

FIG. 8 Elugram and molecular weight distribution diagram of XlinCA (6) FIG. 9 shows a representative slice of a vascular casting performed with ExiTron nano 12000. Scale bar: 1 mm. Kidneys were perfused via the abdominal aorta with 10 ml PBS, 100 ml 4% PFA/1% GA in PBS, 15 ml PBs and 400 μl ExiTron nano 12′000 in 1.6 ml PBS. Renal artery and vein were ligated immediately afterwards, and the kidneys excised and embedded in 6% gelatin with 1% GA in PBS. Kidneys were then scanned on a General Electric Nanotom m with 4.4 μm voxel size. FIG. 9 shows good vascular casting in the outer medulla. However, in several regions in the cortex and inner medulla capillaries are insufficiently filled. Noticeable aggregates in the glomeruli suggest blockage at the afferent arterioles as the causative factor. Attempts at reducing aggregation by dissolving ExiTron nano 12′000 in isoosmolar mannitol solution and pressing them through a 1.2 μm pore syringe filter did not remove all the aggregates and yielded insufficient contrast for capillary imaging.

FIG. 10 A: Left kidney of a 10 month old female C57BL/6J mouse. A number of fluid-filled structures are indicated by green arrows. These structures cannot be captured by previous vascular casting protocols, and were identified as eosinophilic protein-filled casts by subsequent histology. Voxel size 4.4 μm. Scale bar: 1 mm. B: Right kidney of the same mouse. C: Left kidney of an independent 10 month old female C57BL/6J mouse. Fluid-filled structures are indicated by green arrows. Part of the left adrenal gland is unperfused, while the rest is completely perfused (green circle). This region is likely supplied by vessels other than the renal artery and were not perfused due to the ligations applied to the abdominal aorta and superior mesenteric artery. Voxel size 4.4 μm. Scale bar: 1 mm. D: Right kidney of the same mouse. The right adrenal gland is fully perfused.

FIG. 11 Transmission electron microscopy (TEM) images confirm that instillation of the contrast agent does not induce any tissue damage. A: Cortex. Non-contrasted TEM image of part of a glomerulus. Contrast agent is visible as coarsely granular material within the lumen of mesangial capillary loops (CL), and not found in the Bowman's space. Capillary loops are delineated by basement membrane with podocyte foot processes (arrows). Scale bar: 10 μm. B: Cortex. Contrasted TEM image of interstitial cortical capillaries (Cap) containing finely granular contrast agent. PCT—proximal convoluted tubule with intact ciliated epithelial cells. C: Medulla. Contrasted TEM image of interstitial cortical capillaries (Cap) containing moderately granular contrast agent of variable electron density. Tub—tubule with intact non-ciliated mitochondria rich epithelial cell. Scale bar: 10 μm.

FIG. 12 A: Overview image of an HE-stained histological slice containing an improperly perfused region. Pixel size: Downsampled 8× to 1.8 μm. Scale bar: 1 mm. B: Similar slice from the X-ray micro-CT dataset. Pixel size: 4.4 μm. Scale bar: 1 mm C: Magnified view of the improperly perfused region. Glomeruli show considerable numbers of red blood cells, indicating an insufficient initial flushing of the glomeruli as the cause of the collapsed tubuli. Contrast agent is visible in some of the glomeruli along with the red blood cells in both the histology and the X-ray micro-CT dataset, indicating filling of the vessels despite blockage via remaining red blood cells. Pixel size: 227 nm. Scale bar: 100 μm.

### EXAMPLES

### Example 1: Combined Vascular and Tubular Ex Vivo Imaging of Whole Mouse Kidneys

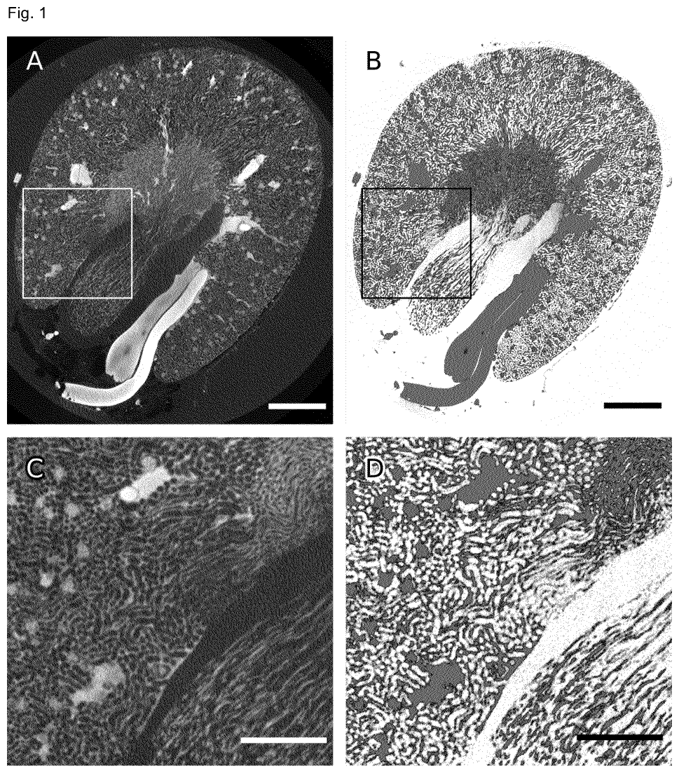

The new protocol allowed the inventors to fill the vasculature of mouse kidneys with contrast agent at lower pressures than what is required for reliable filling with plastic resin-based materials. No water inclusions artifacts or disconnects could be seen, even in kidneys perfused at low flow rates due to imperfect surgery. The inventors acquired micro-CT datasets with 3.3 μm and 4.4 μm voxel sizes with sufficient contrast to distinguish vascular and tubular lumina (FIG. 1). Retention within the vascular lumen and cortical tubular tissue was permanent. The 3.3 μm voxel size dataset was acquired after one month of storage and no reduction in contrast was noticeable.

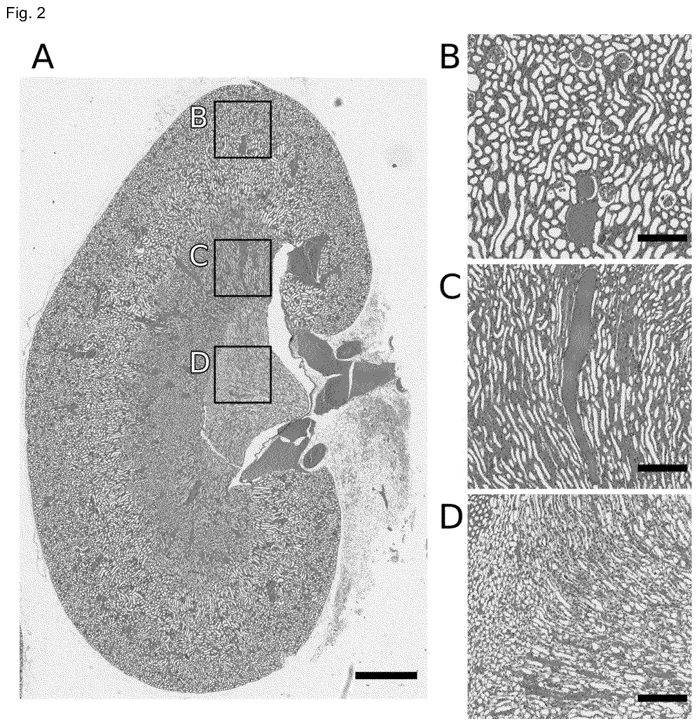

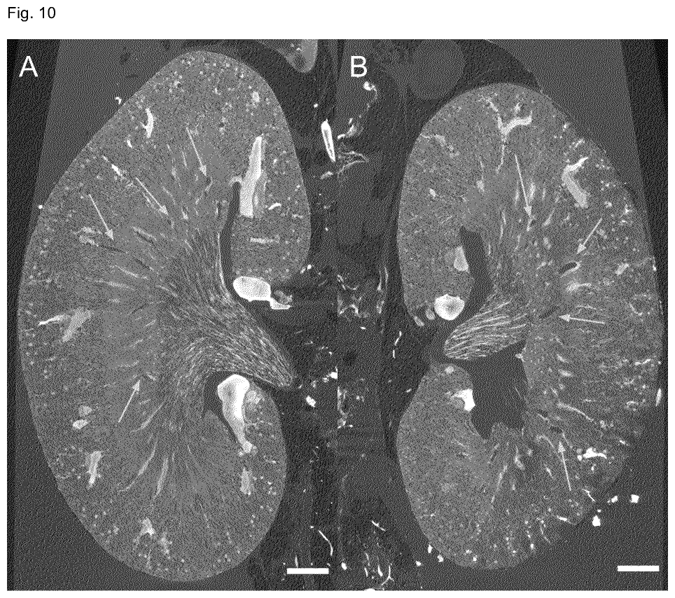

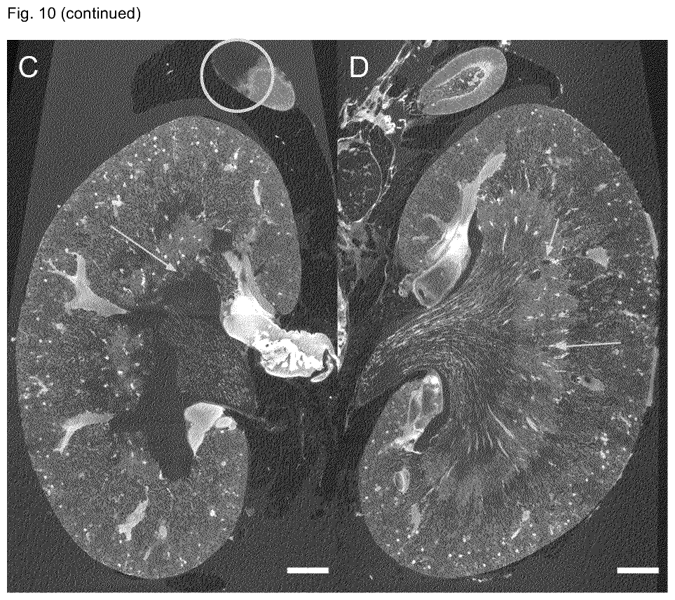

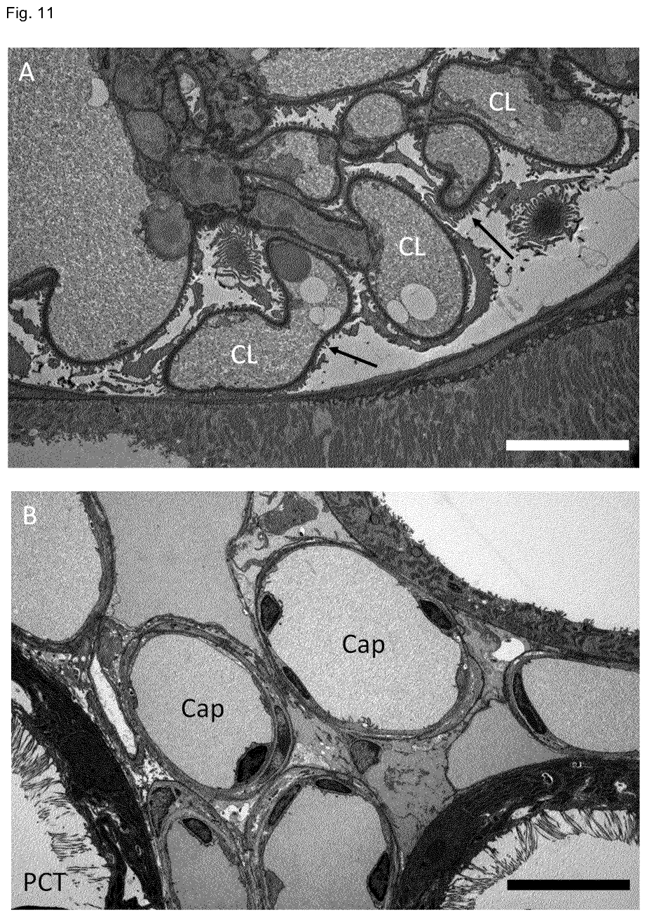

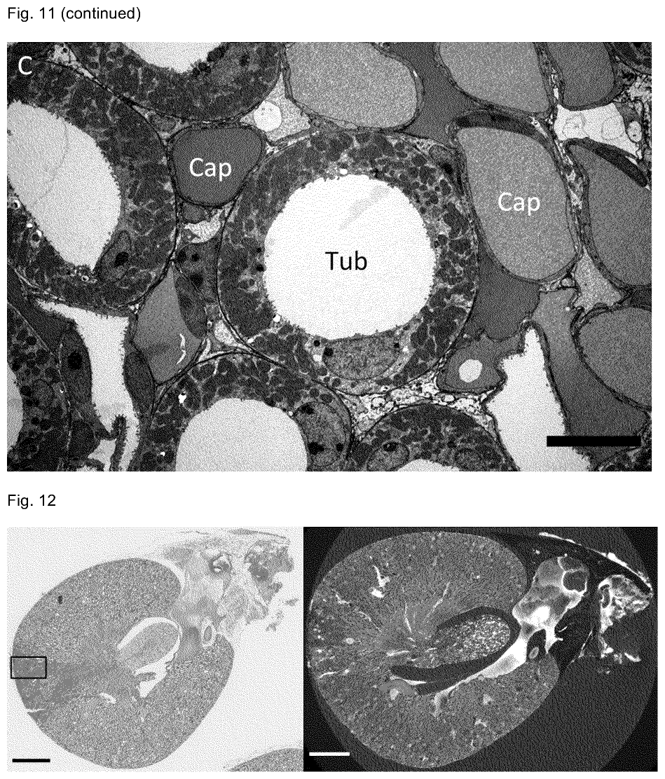

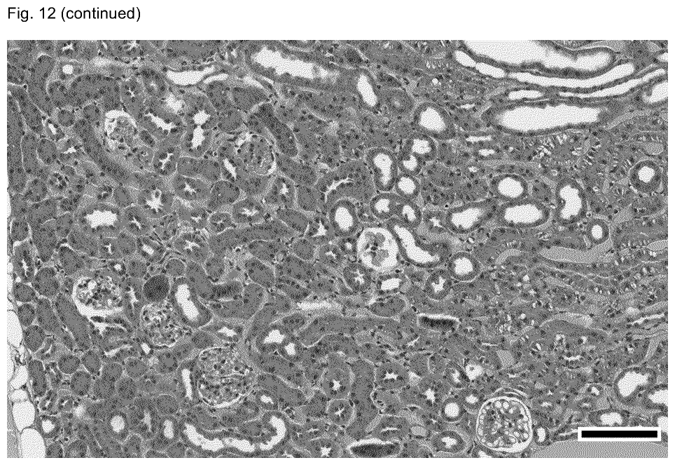

One unexpected discovery was the occurrence of several fluid-filled structures in all mice, despite using healthy mice with non-transgenic background (FIG. 10). These would not have been captured with previous vascular casting methods, which provide no direct means of identifying tissue. Histological examination identified those as eosinophilic protein-filled structures, probably of tubular origin. No tissue damage was found on haematoxylin & eosin stained sections. Cellular ultrastructures were well preserved in transmission electron microscopy images (FIG. 2. or FIG. 11). No sign of osmotic swelling or shrinkage of cells due to the contrast agent could be found. Capillaries were found to be distended, which is explainable by the 150 mmHg of perfusion pressure during fixation. While this is well above resting blood pressure, it represents the upper end of blood pressure during physical activity. The inventors could identify microscopic gas bubbles in the polymerized contrast agent hydrogel, which were not visible in the X-ray micro-CT datasets, as they were typically smaller than the resolution limit. They therefore have no noticeable impact on the final data, and are much smaller than gas bubbles common in plastic-resin based vascular casting. Nevertheless, the contrast agent solution should be degassed extensively prior to perfusion if higher resolution scans are planned. The inventors also evaluated a kidney with imperfect perfusion surgery, where a region of the kidney cortex was not filled with contrast agent. Presence of erythrocytes in the badly perfused region (FIG. 12) suggests that the lack of filling is caused by insufficient flushing of the blood vessels prior to contrast agent injection, and thus not a contrast agent-related issue.

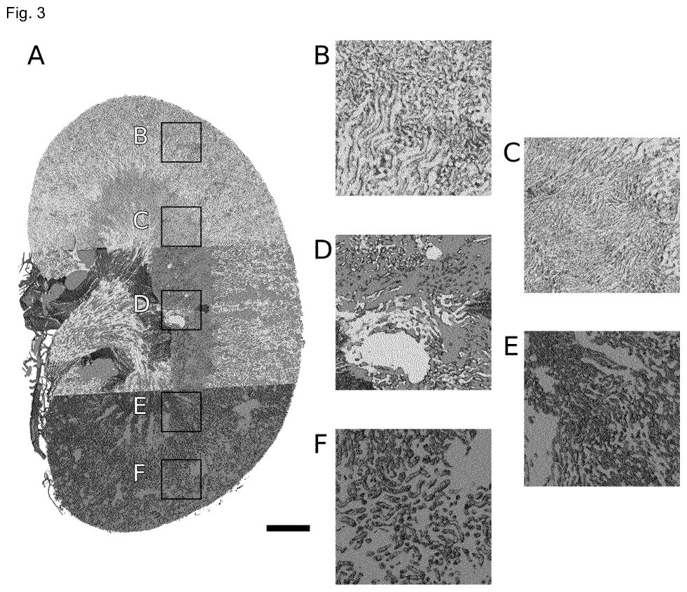

The inventors were able to extract the vascular and tubular lumina from the 3.3 μm voxel size X-ray micro-CT dataset with Fiji/ImageJ in a semiautomatic workflow as binary masks. The inventors visualized these binary masks with commercial software (FIG. 3). They further allowed a variety of automated quantifications. For example, the inventors evaluated blood vessel density (a measure used to quantify capillary rarefaction (Ehling et al., 2016)) by calculating the number of voxels of the masks. The volume of the segmented blood vessel lumina was 65.6 mm³, of the tubular lumina 58.5 mm³, of the tissue 42.6 mm³ and of the whole kidney 166.7 mm³, resulting in a vessel density of 39%.

Line probe intersection can be used as in stereology to measure surface area. The inventors used MorphoLibJ in 13 directions to do this fully automatically. Surface areas were 8433 mm² and 8775 mm² for the segmented blood vessels and tubules, respectively. This information can be used, for example, to quantify the diffusion of oxygen across the blood vessel walls, which is proportional to the surface area (Ngo et al., 2014). The inventors are certain to have underestimated the surface areas, as they do not include the surface area within the glomeruli or the full area of the vascular bundles due to the limited resolution of the imaging. The numbers reported represent therefore a biased measure, which may still be useful for comparative quantification.

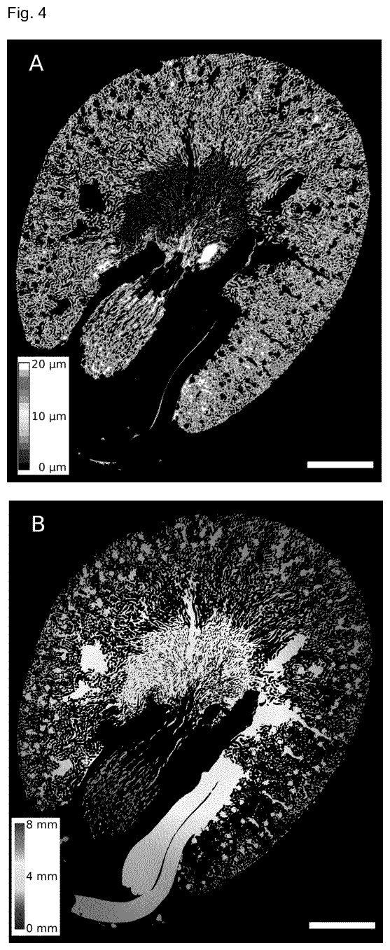

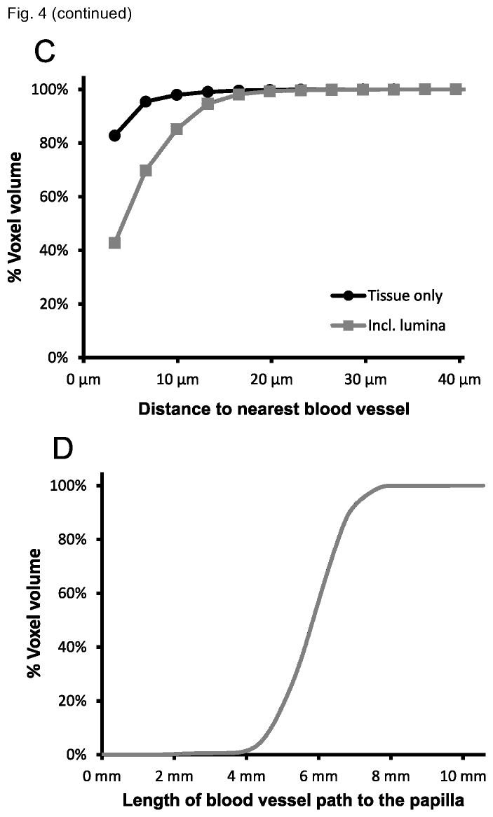

The inventors then calculated the 3D Euclidean distance of each voxel to the nearest blood vessel, which represents the minimal diffusion distance of a given location of the kidney to the nearest source of oxygen and nutrients (Borgefors, 1996). This can be used to quantify the amount of tissue that is insufficiently supplied (Prommer et al., 2018). The inventors evaluated these distances for the entire space of the kidney devoid of blood vessels, creating a distance map (FIG. 4A). The inventors then evaluated the distribution of the distances within the kidney by taking into account either the whole non-blood vessel space, or only the renal tissue. 43% of the considered non-blood vessel space is contained within the first neighboring voxel adjacent to a blood vessel if the whole space, including the tubular lumina, is evaluated. However, if tubular lumina are excluded and only tissue is considered, 83% of the tissue is contained within the first voxel. This result demonstrates the potential for misinterpretation when evaluating the diffusion distances of oxygen or other nutrients based solely on vascular data.

The inventors then selected manually a marker point at the papilla of the kidney in the blood vessel segment and calculated the lengths of the shortest path along the blood vessels for every voxel to that marker. In the resulting false-color map (FIG. 4C), the inventors could identify 4 mm as the approximate cut-off point for the path distance at which the blood vessels exit the inner medulla and enter the outer medulla. Calculating the cumulative distribution function revealed that only 1.5% of the blood vessel volume is contained in the inner medulla. In principle, this quantification would allow measurement of the path length of arbitrary blood vessels or tubules (Lantuejoul and Beucher, 1981). Since the inventor's vascular and tubular masks contain a variety of artificial shortcuts introduced by the limited resolution, the path lengths shown here are not reliable beyond the inner medulla.

Methods

X-Ray Contrast Agent Synthesis

An acryloyl group was added to the amine of 5-amino-2,4,6-triiodoisophthalic acid. The resulting compound was polymerized via reversible addition-fragmentation chain-transfer polymerization (Chiefari et al., 1998; Lai et al., 2002) to a molecular weight of approximately 20 000 g/mol. Ethylenediamine was coupled with the carboxylic acid groups of the polymer to add free amine groups, enabling aldehyde fixation. To increase the size of the polymer, it was pre-crosslinked with a small amount of glutaraldehyde and dialyzed against a 100 000 MW membrane. (see also example 2)

Mouse Husbandry

Female C57BL/6J mice were purchased from Charles River Laboratories and Janvier Labs and were kept until 7 months old with ad libitum access to water and standard rodent food (Kliba Nafag 3436).

Abdominal Aorta Perfusion

Mice were anaesthetized with Ketamine/Xylazine, and kidneys were perfused retrogradely via the abdominal aorta (Czogalla et al., 2016) with a 21 G butterfly needle connected via a 2.5 m long silicon tube to a reservoir providing 150 mmHg of hydrostatic pressure. The kidneys were flushed with approximately 10 ml of phosphate-buffered saline (PBS) and fixed with 100 ml 4% formaldehyde/1% glutaraldehyde/PBS. Remaining aldehydes were flushed out with 20 ml PBS and quenched with 50 ml glycine solution (5 mg/ml in PBS), then flushed again with another 40 ml PBS. All perfused solutions were kept at 37° C. 4 ml of X-ray contrast agent solution (75 mg l/ml) were perfused using a 10 ml syringe, actuated with a constant weight to provide 150 mmHg of pressure. The abdominal cavity was then filled with 4% glutaraldehyde/PBS to crosslink the contrast agent, and the kidneys removed afterwards and kept in 4% glutaraldehyde/PBS. These solutions were kept at room temperature.

Kidneys were mounted in 1% Agar/PBS in either standard 1.5 ml Eppendorf tubes or 0.5 ml PCR tubes for scanning, depending on their size.

X-Ray Micro-CT Image Acquisition

The X-ray micro-CT images were acquired with a General Electric Phoenix Nanotom m, equipped with a tungsten target and diamond window. Acceleration voltage was set to 60 kV, current to 310 μA. 1440 projections were acquired per height step with a GE DXR detector with a 3052×2400 pixel array with 0.5 s exposure time. Four height steps were required for each kidney. Kidneys mounted in Eppendorf tubes were scanned with 4.4 μm isotropic voxel size. 3 frames per projection were recorded and averaged, resulting in a scan time of approximately 3 h per kidney. The kidney mounted in the PCR tube was scanned with 3.3 μm isotropic voxel size with 12 frames per projection averaged, resulting in 10 h of scan time.

Reconstruction was performed with the manufacturer's GE phoenix datos|x software.

Histological Examination

Fixed kidneys were trimmed (midline longitudinal or cross section) and paraffin wax embedded. Consecutive sections (3-5 μm) were stained with hematoxylin-eosin (HE) and the periodic acid Schiff reaction for histological examination, or were deparaffinized and left unstained for assessment of fluorescence. Slides were photographed with a Nikon Eclipse Ni-U microscope with digital camera and scanned using a digital slide scanner with 40× magnification (NanoZoomer-XR C12000, Hamamatsu, Japan).

Transmission Electron Microscopy Examination

A slice of fixed kidney (midline cross section) was trimmed and embedded in epoxy resin. Toluidine blue-stained semithin (1.5 μm) sections were prepared to select areas of interest for the preparation of ultrathin (75 nm) sections that were either directly viewed with a Philips CM10 microscope, operating with a Gatan Orius Sc1000 digital camera (Gatan Microscopical Suite, Digital Micrograph), or were contrasted with lead citrate and uranyl acetate and viewed subsequently.

Example Segmentation and Quantification

The inventors segmented the 3.3 μm dataset using the free Fiji/ImageJ (Schindelin et al., 2012; Schneider et al., 2012) software with the MorphoLibJ (Legland et al., 2016) and 3D ImageJ Suite plugins (Ollion et al., 2013) installed. The inventors applied a 3D Gauss filter with σ=1 to the cropped dataset and extracted the blood vessels by setting manual thresholds. Different thresholds had to be applied for the first and subsequent height step of the scan. The inventors then created a rough kidney mask by first setting another, lower threshold, and removed areas of contrast outside the kidney by performing erosion and connected component analysis with the MorphoLibJ plugin (Legland et al., 2016). This mask was then applied to the blood vessel segment.

The blood vessel segment was then transformed into a more refined kidney mask by dilation, 3D hole filling using the 3D ImageJ Suite plugin (Ollion et al., 2013) and subsequent erosion. This mask was combined with thresholding of the kidney water background to receive the tubular lumen. Any remaining volume within the mask that was neither part of the blood vessels nor part of the tubular lumen was declared as kidney tissue.

The inventors used the MorphoLibJ plugin to quantify vessel and tubular volumes by simple voxel count and surface by the intersection of line probes in 13 directions. The inventors then selected manually a marker point at the papilla of the kidney in the blood vessel segment and calculated the geodesic distance map using the same plugin. The Euclidean distance map and all histograms were calculated using default Fiji/ImageJ functions.

Image processing was performed on a workstation equipped with 256 GB RAM and two Intel Xeon E5-2670 processors. 3D computer graphic images were rendered with VGStudio Max 2.1 (Volume Graphics) on a workstation equipped with 128 GB RAM and an Intel Xeon E5-2620 v3 processor.

### Example 2: Vascular Imaging of a Mouse Brain Hemisphere and of an Entire Mouse

Design of the new cross-linkable polymeric contrast agent XlinCA was designed to fulfil a specific set of criteria to resolve issues encountered in ex vivo vascular imaging with current contrast agents:
Highly water-soluble, to avoid incomplete vascular filling and interrupted vessel segments.
High molecular weight, to prevent the immediate leakage through blood vessel walls.
Cross-linkable, to prevent leakage over time.
High X-ray attenuation coefficient, to reduce the required contrast agent concentration and reduce viscosity and osmolarity.

The following chemical formula represents the cross-linkable polymeric X-ray contrast agent

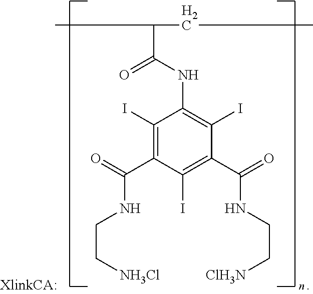

Highly water-soluble X-ray contrast agents inherently avoid issues with water inclusions and high flow resistance of the hydrophobic vascular casting resins, as well as the sedimentation and aggregation problems of nanoparticle suspensions.

High molecular weight blood pool contrast agents cannot pass through blood vessel walls, and retain contrast over much longer time periods than standard angiography contrast agents.

XlinCA was, therefore, designed to provide a polymer with a molecular weight above 65 kDa, which corresponds to the molecular weight of serum albumin, the most abundant protein in the blood.

Cross-linkable contrast agents avoid the loss of contrast over even longer time scales by covalently linking the contrast agent to itself and to the tissue. Aldehydes are used in tissue fixation to cross-link proteins and are thus well-compatible with tissue preparation protocols. XlinCA was designed to contain free primary amine groups as targets for glutaraldehyde fixation, which enables cross-linking via imine formation (Cheung, et al. 1982 and Migneault et al. 2004).

High X-ray attenuation coefficients are achieved by increasing the electron density through the inclusion of heavy atoms, such as iodine, barium, gadolinium, gold or lead. Iodine was chosen due to the low cost, synthetic availability and low toxicity. The higher the content of iodine in the contrast agent is, the lower the concentration of the contrast agent has to be to achieve a given contrast-to-noise ratio. Since the high molecular weight of a polymeric contrast agent could lead to high viscosity, lowering the required contrast agent concentration is crucial to keep the final contrast agent solution easily perfusable through the vascular system.

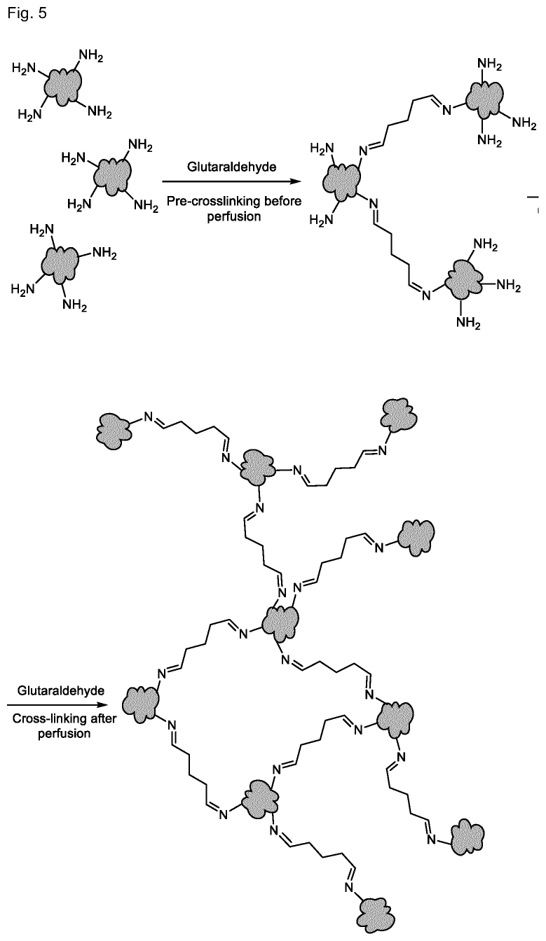

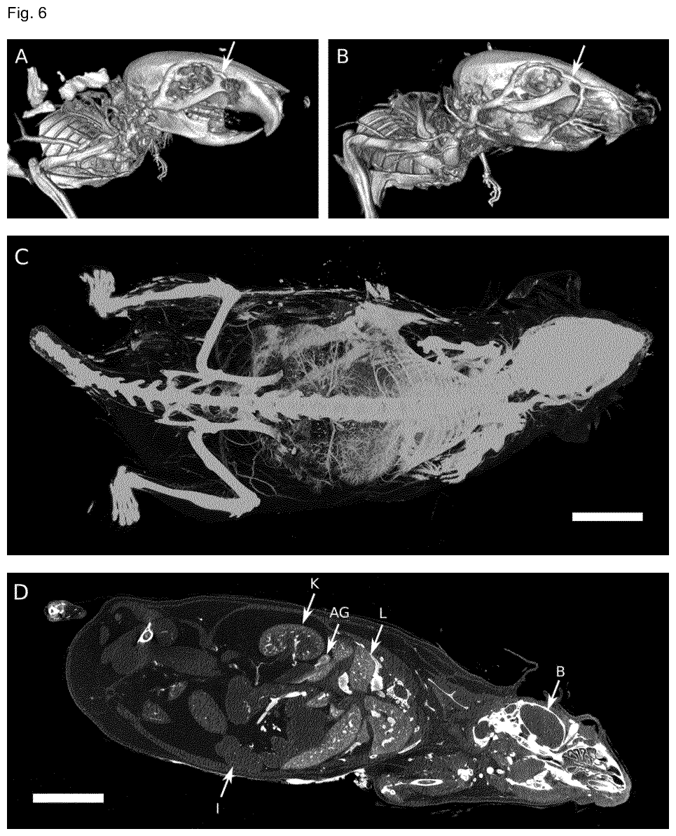

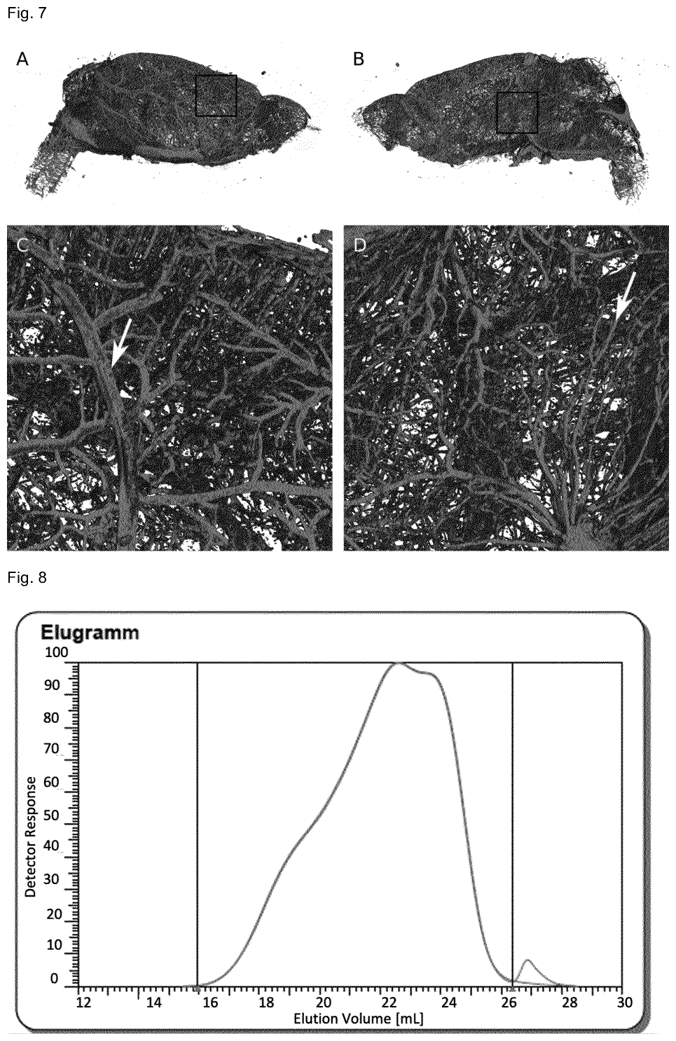

After considering all the above criteria, the inventors designed the cross-linkable polymeric contrast agent XlinCA represented schematically in FIG. 5. The theoretical iodine content is 49.5%, which is comparable to standard small molecule angiography iodine contrast agents and considerably higher than what could be achieved by other typical approaches in increasing molecular weight, such as linkage to polyethylene glycol (PEG).

Synthesis of the Contrast Agent

The contrast agent was synthesized through a multi-step process, see Scheme 1. Starting from the commercially available 5-amino-2,4,6-triiodoisophthalic acid 1, the acryloyl group was added through reaction with a typical acylating agent, namely acrylic anhydride with a catalytic amount of sulfuric acid. The reaction was straightforward, giving acrylamide 2 in good yield. Multiple attempts to synthesize compound 1 with the help of the cheaper acryloyl chloride reagent were unsuccessful.

The key step in this synthesis was the polymerization of 2 to give polymer 3 by reversible addition-fragmentation chain transfer (RAFT) polymerization. In comparison to conventional free radical polymerization, RAFT polymerization employs chain transfer agents (RAFT agents or CTA), which reversibly stop chain propagation and can initiate the growth of a new chain. This results in a living free radical polymerization, which reduces the probability of chain termination by reducing the number of simultaneously propagating chains, and equilibrates the growth amongst different chains. These properties allow for the synthesis of polymers with controlled high molecular weight and low polydispersity.

The choice of the appropriate RAFT agent is crucial for a successful polymerization. There is a wide range of RAFT agents available for mostly all classes of monomers polymerized by free radical mechanisms. Trithiocarbonate 8 was chosen for polymerization of 2 due to the reported compatibility with various acrylamide derivatives.

The inventors found that dimethylformamide (DMF) is the most suitable solvent for RAFT polymerization of 2. It provides high solubility of both starting materials and polymeric products and allows a high conversion degree of the monomers. Dimethylsulfoxide (DMSO) also provides good solubility, but does not allow for a good conversion of 2 to 3. The optimal reaction temperature was 70° C. Lowering this temperature significantly slowed down the reaction and higher temperatures led to lower polymer yields.

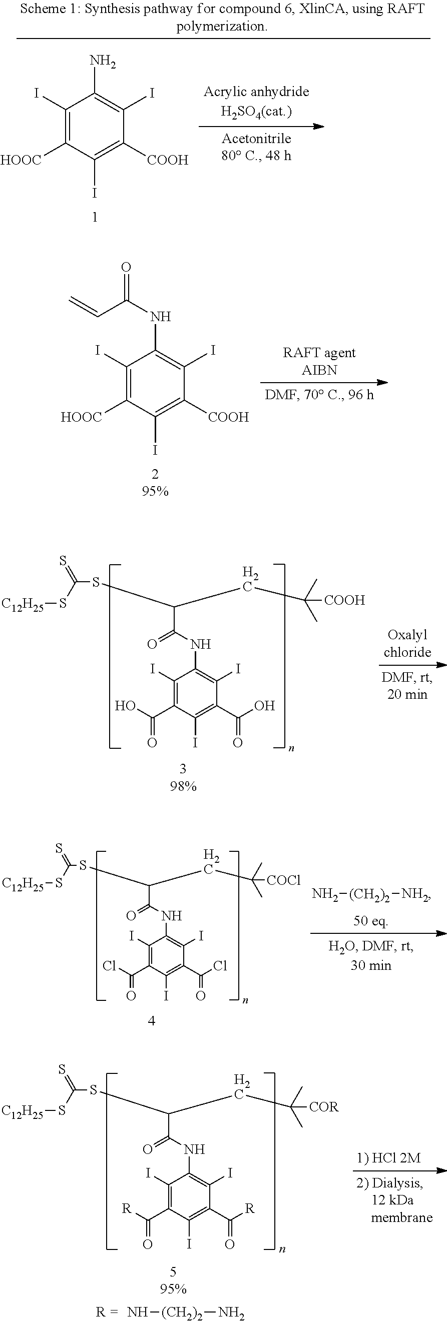

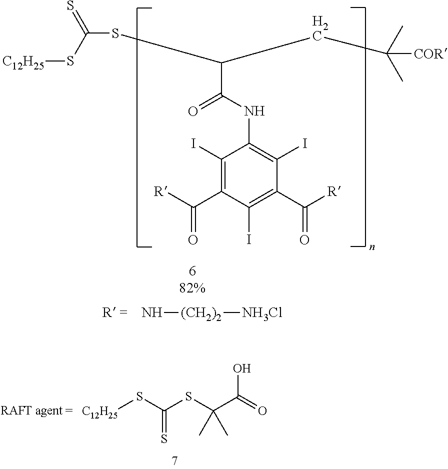

The inventors aimed to obtain a polymer with a molecular weight above 65 000 g/mol, the molecular weight of blood serum albumin, in order to prevent its diffusion through the blood vessel walls. The average molecular weight of the synthesized polymer increases with increasing ratio of monomer to RAFT agent. The optimal ratio of monomer:RAFT agent:radical initiator was found to be 400:2:1. Increasing this ratio further made the reaction extremely vulnerable to stirring or mechanic vibration, leading to a higher tendency towards precipitation and aggregation of the product before completion of the reaction.

The proton nuclear magnetic resonance (NMR) spectrum was measured at the end of the reaction to calculate the conversion degree of the polymerization process, which resulted in 78%. It was determined via the ratio of the integral of the methine proton signal at 2.54 to 2.60 ppm to half of the integral of the carboxylic group proton signal at 13.4 to 14.5 ppm.

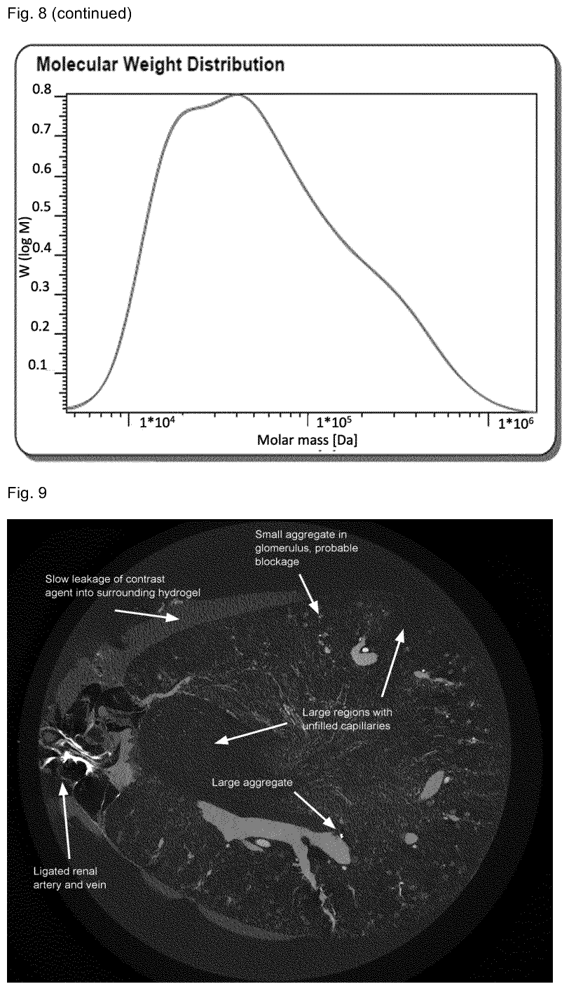

The molecular weight of 3 could not be measured with gel permeation chromatography (GPC), due to low solubility, even in DMF, and tendency of the polymer towards aggregation. However, the molecular weight and polydispersity index of 3 could be indirectly estimated from the GPC result of the final product 6, which is water soluble and stable against aggregation due to presence of ionic charges in the side chains of the polymer. Based on the ratio of the molecular weights of the monomers and the average molecular weight of 6 (FIG. 8), we calculated 30 400 Da for the average molecular weight of 3.

Ethylenediamine was added to the polymer 3 after activation of the carboxylic groups with oxalyl chloride. Large excess of ethylenediamine was used to prevent crosslinking of the acyl groups. The obtained 5 was easily dissolved in diluted hydrochloric acid solution to give the contrast agent XlinCA (6), which is water soluble and can be cross-linked with aldehydes due to the presence of the amine groups.

Pre-Crosslinking of Contrast Agent

The ability to be cross-linked not only enables polymerization of XlinCA after injection in the vasculature, but also allows for the synthesis of arbitrarily large contrast agent molecules by pre-crosslinking with glutaraldehyde (FIG. 5). In order to increase the molecular weight from 30 kDa to above the required threshold of 65 kDa, XlinCA was pre-crosslinked with different amounts of 25% glutaraldehyde prior to perfusion into the mouse. The optimized amount was found to be 30 μL of 25% glutaraldehyde solution per 1 g of XlinCA dissolved in 4 mL water. This ratio led to a limited increase in viscosity, which indicated the increase in molecular weight of the polymer by pre-crosslinking. Higher amounts of glutaraldehyde led to gelation of the contrast agent, either immediately or during the dialysis process. The pre-crosslinked contrast agent was dialysed with a 100 kDa dialysis membrane, lyophilised and stored until ready for perfusion into the blood vessels. After pre-crosslinking, the contrast agent should be used within a week after lyophilisation, as it becomes slowly insoluble over time. This may be caused by further cross-linking by residual aldehydes after dialysis.

After the contrast agent was administered into the vasculature, further crosslinking of the contrast agent with glutaraldehyde led to gelation of the contrast agent, preventing any leakage and loss of contrast over time (FIG. 5).

Imaging of Whole Mouse Vasculature

Mice perfused with vascular casting resin PU4ii and polymeric contrast agent XlinCA were compared based on low-resolution μCT scans with a voxel size of 80 μm. In the PU4ii-perfused mouse, numerous water inclusions and gas bubbles could be observed in the descending aorta, vena cava and larger vessels in the kidneys and the liver. Correspondingly, one liver lobe, adrenal glands and part of the kidneys were only partially filled with PU4ii. Certain large blood vessels, such as the naso-frontal vein and anterior vein, remained completely devoid of PU4ii (FIG. 6A), while their counterparts in the mouse perfused with XlinCA were filled in their entirety (FIG. 6B). To further evaluate completeness of vessel filling, the XlinCA-perfused mouse was scanned with a voxel size of 20 μm. Brain, heart, lungs, liver, kidneys and adrenal glands appeared well-perfused, see FIGS. 6C and D. No discontinuous vessel segments could be found, as was expected for a water-soluble compound.

Vascular filling with XlinCA was not entirely complete. Small vessels posterior to the kidneys appeared not well defined. Parts of the capillaries were missing in the renal medulla of the kidney and the spleen. Leftover blood seen in the histological examination indicated that this was caused by incomplete flushing of blood prior to contrast agent injection, and thus not due to any contrast agent-related property.

Imaging of the Brain Vasculature

The brain of the XlinCA-perfused mouse was removed and the right brain hemisphere was scanned with a voxel size of 4.4 μm, as the smaller field of view allowed for scans with approximately twice the resolution compared to a full brain. There were no large areas of missing vasculature as was reported for optimized Microfil perfusions (Ghanavati et al., 2014). No vessel discontinuities through insufficient filling or gas bubbles could be identified, as with the results in the whole mouse.

Independent of the choice of vascular casting agent, non-optimized transcardial perfusion techniques cannot entirely outperform optimized perfusion techniques in specific organs. On equal terms, however, XlinCA provided more complete and reliable filling of multiple organs in an entire mouse with a simple transcardial injection, without requiring clamping or ligation of the descending aorta and vena cava. The procedure is accordingly much simpler to perform and allows multiple organs to be harvested and used in the analysis of the vasculature, reducing the number of animals required and reducing the variance in multi-organ correlation studies. Factors such as injection volume and flow rate did not have to be optimized, as cross-linking can be initiated at any time after perfusion is complete. In contrast, Microfil polymerizes within approximately 20 minutes ((Ghanavati et al., 2014), limiting the perfusion volume before viscosity and thus resistance to flow increases. With the time constraints removed, flow rate and thus perfusion pressure are no longer factors that require optimization. Perfusion pressure in our experiment was 150 mmHg, which is consistent with the pressure used in the transcardial perfusion reported by Chugh et al. 2009, but lower pressures can be used without risk of premature polymerization and resulting incomplete filling. Higher perfusion pressures still are of advantage in the prior perfusion steps required for flushing the vasculature of remaining blood, however.

Methods

General

Unless otherwise stated, all chemicals were of reagent grade and purchased from Sigma-Aldrich. All solvents were of analytical grade. Dialysis tubings were purchased from Sigma-Aldrich (cellulose, 12 kDa cut-off) and from Spectrum (cellulose ester, 100 kDa cut-off). The ¹H NMR and ¹³C NMR spectra were recorded on a Bruker 400 MHz spectrometer. Low resolution mass spectral analysis was performed with a Waters AQUITY-Bruker UPLC-MS system. Analytical gel permeation chromatography was performed by analytical service of PSS Polymer (Mainz, Germany) using columns PSS-NovemaMax_F 5 μm: Guard+30 Å+1000 Å+1000 Å with UV/VIS and differential refractometer RID detectors. Aqueous solution of 0.1 M NaCl/0.1 Vol.-% TFA was used as eluent. The average molecular weight and the molecular weight distribution of the samples were calculated based on standard calibration of pullulan.

Synthesis and characterization of acrylic anhydride were in accordance with the literature Jian et al. 2015). General synthesis procedure and characterization of intermediates and the final product XlinCA 6 are described below.

Mouse Husbandry

C57BL/6J mice were purchased from Charles River Laboratories and Janvier Labs and were housed in individually ventilated cages in 12 h light/dark cycles with ad libitum access to water and standard rodent food (Kliba Nafag 3436). All animal experiments were approved by the veterinary office of the canton of Zurich.

Pre-Crosslinking of Contrast Agent

Contrast agent XlinCA (5 g) was dissolved in water (20 mL) then 150 μL of an aqueous glutaraldehyde solution (25%) were added and mixed well. The mixture was left to rest at room temperature for 20 min. Afterwards, 30 mL of water were added, and the solution was dialysed through a 100 kDa dialysis membrane against NaCl solution (5 L, 0.2%), changing the solution after 3 h, 8 h and 24 h; then against deionized water for 6 h. The solution was centrifuged to remove all insoluble particles and lyophilized to give 4 g of a solid, pre-crosslinked contrast agent.

Transcardial Whole Body Perfusion with Polymeric Contrast Agent

A 9 month old mouse was euthanized with ketamine/xylazine. The chest cavity was opened, a blunted 21 G butterfly needle inserted into the left ventricle and the right atrium cut as an outlet. Blood was flushed out with approx. 10 mL of phosphate-buffered saline (PBS) and the mouse fixed with 100 mL 4% formaldehyde and 1% glutaraldehyde in PBS. The aldehydes were flushed out with 50 mL PBS and quenched with 50 mL 0.5% glycine in PBS. The mouse was finally flushed with 25 mL PBS and perfused with 14 mL contrast agent solution filtered through a 1.2 μm pore syringe filter (2.7 g of pre-crosslinked above contrast agent in 14 mL H₂O, 100 mg iodine/ml prior to filtering). To close the outlet, 4% glutaraldehyde in PBS was dripped onto the heart to initiate cross-linking. The entire mouse was subsequently immersed in 500 mL 4% glutaraldehyde in PBS.

Transcardial Whole Body Perfusion with Vascular Casting Resin PU4ii

The left ventricle was cannulated as above, and the blood was flushed out with approx. 10 mL of PBS and the mouse fixed with 100 mL 4% formaldehyde in PBS. Vascular casting was performed using a mixture of 3.7 g 1,3-diiodobenzene (Sigma-Aldrich, USA) with the vascular casting resin PU4ii (vasQtec, Switzerland), which consists of 10 g 2-butanone, 10 g PU4ii resin and 1.6 g PU4ii hardener. The final contrast agent concentration of the PU4ii mixture was 110 mg iodine/ml.

X-Ray μCT Scans

For the low-resolution comparison scans, the heads of the PU4ii- and XlinCA-perfused mice were scanned with 80 μm voxel size using a QuantumFX in vivo μCT scanner (PerkinElmer, USA) with an acceleration voltage of 70 kV and a tube current of 200 μA. High-resolution scans were performed on a Nanotom m μCT scanner (General Electric, USA) using an X-ray tube with water-cooled tungsten target set to an acceleration voltage of 60 kV and a tube current of 310 μA. 1440 projections per height step were acquired with 0.5 s exposure time using a scintillator-coupled flat-panel detector. The mouse was removed from the fixation solution, mounted in a plastic cup using polyurethane foam and scanned with 20 μm voxel size. The brain was excised, cut in the middle with a razor blade and the right brain hemisphere was embedded in 1% agar in a 1.5 mL centrifugation tube and scanned with 4.4 μm voxel size.

The whole mouse was visualized using Arivis4D 2.12.4 (Arivis, Germany) and the brain hemisphere was visualized using VGStudio Max 2.1 (Volume Graphics, Germany).

### Synthesis of 2,4,6-triiodo-5-(prop-2-enamido)benzene-1,3-dicarboxylic acid (2) (See Scheme 1)

To a suspension of 5-amino-2,4,6-triiodobenzene-1,3-dicarboxylic acid (1) (20 g, 35.8 mmol) and concentrated sulfuric acid (0.04 mL) in acetonitrile (40 mL), acrylic anhydride (12 mL, 107 mmol) was added drop-wise while the reaction mixture was being cooled in an ice bath. The reaction mixture was stirred at 80° C. for 36 h until no 5-amino-2,4,6-triiodobenzene-1,3-dicarboxylic acid (1) was detected by UPLC. The mixture was cooled down to room temperature, then filtered under vacuum and washed with acetonitrile (20 mL). The solid was dried in high vacuum for two days to yield 20.8 g (95%) of a white product. UV-Vis spectrum (methanol, X; nm): 243; IR [KBr, cm⁻¹]: 3247, 3000, 1725, 609, 508; ¹H NMR (400 MHz, DMSO-d₆): 5.83 (dd, J²=1.8 Hz, J³cis=10.2 Hz, 1H), 6.32 (dd, J²=1.7 Hz, J³trans=17.2 Hz, 1H), 6.44 (dd, J³cis=10.2 Hz, J³trans=17.1 Hz, 1H), 10.22 (s, 1H), 14.1 (s, broad, 1H). ¹³C NMR (101 MHz, DMSO-d₆): 169.82, 163.82, 127.96, 131.53, 143.79, 149.57, 98.55, 87.74. ESI-MS: m/z=613.74 [M+H]+; Elemental analysis calcd (%) for C₁₁H₆₃NO₅: C, 21.56; H, 0.99, N, 2.29; found: C, 21.75; H, 0.93; N, 2.36.

### Synthesis of Poly(2,4,6-triiodo-5-(prop-2-enamido)benzene-1,3-dicarboxylic acid) (3) (See Scheme 1)

2,4,6-triiodo-5-(prop-2-enamido)benzene-1,3-dicarboxylic acid (2) (25.12 g, 40 mmol) was dissolved in DMF (40 mL). Then, 2-(dodecylthiocarbonothioylthio)-2-methylpropionic acid (7) (72.8 mg, 0.2 mmol) and AIBN (16.4 mg, 0.1 mmol) were added. The solution was degassed by three freeze-evacuate-thaw cycles and transferred to an oil bath preheated at 70° C. under nitrogen flow. The polymerization was carried out for 96 h under slow stirring and then was quenched by cooling in an ice bath under atmospheric air for 30 minutes (conversion degree 78%). DMF was removed on a rotovap at 10 mbar, 50° C. and the product was dried under high vacuum for 2 days to give polymer 3 (24.6 g, 95%) as a yellowish solid. ¹H NMR (400 MHz, DMSO-d₆): 5.82 (dd, J²=1.8 Hz, J³cis=10.2 Hz, 0.22H), 6.30 (dd, J²=1.7 Hz, J³trans=17.2 Hz, 0.26H), 6.44 (dd, J³cis=10.2 Hz, J³trans=17.1 Hz, 0.22H), 10.22 (s, 0.26H), 14.01 (s, broad, 2H). Elemental analysis calcd (%) for C_{15.35}H_{16.15}I₃N_{2.45}O_{6.45} (M+1.45DMF): C, 25.65; H, 2.26; N, 4.77 Found: C, 25.57; H, 2.56; N, 4.41.

Synthesis of XlinCA (6) (See Scheme 1)

The polymer 3 was re-dissolved in 40 mL of DMF before the oxalyl chloride-DMF adduct was added in small portions. The oxalyl chloride-DMF adduct was synthesized by adding oxalyl chloride (15 mL) dropwise over 15 min to a solution of 40 mL of DMF in 300 mL of DCM, while the reaction was being cooled in an ice bath. After 15 min, DCM was evaporated giving the mixture of adduct in DMF, then the whole mixture was used to treat the polymer 3.

The reaction mixture was stirred at room temperature for 30 min. Afterwards, the solution was added quickly to 500 mL of water to precipitate the product. The precipitate was filtered, washed with 100 mL of water, and dried under high vacuum overnight to give the chlorinated polymer 4.

Chlorinated polymer 4 was dissolved in DMF (100 mL) and the solution was added quickly to the ice-cold mixture of ethylene diamine (100 mL) and water (100 mL) under vigorous stirring. After 30 min, the solvents were evaporated in vacuo. Water (50 mL) was added to the residue and the mixture was lyophilized under high vacuum to give 5 (23.4 g, 95%). 5 was dissolved in HCl solution (100 mL, 2 M). The solution was dialyzed with a 12 kDa membrane against NaCl solution (10 L, 0.2%), changing the solution after 3 h, 8 h and 24 h; then against deionized water (10 L) for 6 h more. Afterwards, pH was readjusted to 7 by adding NaOH 1M solution and the solution lyophilized to give the final product XlinCA 6 (19.2 g, 82%), GPC (Mₙ=33700, PDI=3.16).

GPC Measurement of Contrast Agent XlinCA (6) (See FIG. 8)

GPC measurements were done by PSS Polymer Standards Service GmbH, Mainz, Germany.

Sample preparation. About 2 mg of each sample were weighed in on an analytical balance. 2 mL of eluent were added to the sample and left to dissolve at room temperature. After 2 hours, the sample was completely dissolved and could be measured. The sample solution was not filtrated before the measurement and 100 μL were injected by an autosampler.

Calibration and Calculation. Pullulan-standards with different molecular weights were analyzed first in order to get a calibration curve. The calculations of the average molecular weights and the molecular weight distribution of the samples were done by the so-called slice by slice method based on the pullulan-calibration.

TABLE 1
Calculation of the average molecular weights and the molecular weight distribution.

| | | | | PDI | | | |
|---|---|---|---|---|---|---|---|
| Detector | Mₙ/Da | M_{w}/Da | M_{z}/Da | (=M_{w}/Mₙ) | Vₚ/mL | Mₚ/Da | Area |
| UV@230 nm | 33700 | 107000 | 324000 | 3.16 | 22.58 | 39900 | 2686.9200 |
| RID | 33400 | 105000 | 325000 | 3.16 | 22.60 | 39400 | 16.6182 |

Mₙ: Number average molecular weight,
M_{w}: Weight average molecular weight,
M_{z}: Size average molecular weight,
PDI: Polydispersity index,
Vₚ: Elution volume at peak maximum,
Mₚ: Molecular weight at the peak maximum,
Area: Total area under elugram.

### Example 3: Further Monomers

A polymer according to the invention may be obtained by the reaction shown in Scheme 3.

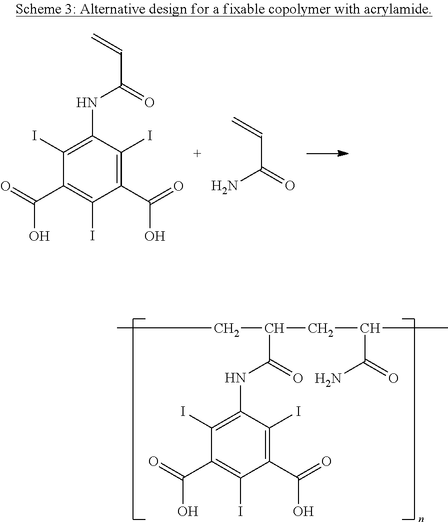

A polymer according to the invention with increased water solubility is

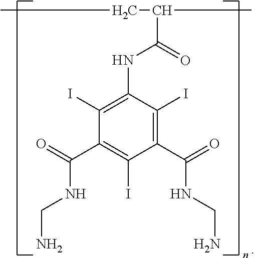

A polymer having a peptide backbone is

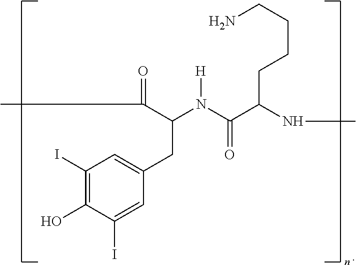

Cleavage of the RA-Adduct:

The sulfur-containing RA-adduct of the polymers of the invention RI-[M]n-RA and FT-[M]n-RA can be cleaved using the methods described by Chong et al. (2007), Moughton et al (2009) or Jesson et al (2017).

### REFERENCES

Borgefors, G., 1996. On Digital Distance Transforms in Three Dimensions. Computer Vision and Image Understanding 64, 368-376.
Cheung, D. T. & Nimni, M. E. Mechanism of Crosslinking of Proteins by Glutaraldehyde.1. Reaction with Model Compounds. Connect. Tissue Res. 10, 187-199 (1982). Chiefari, J., Chong, Y. K. (Bill), Ercole, F., Krstina, J., Jeffery, J., Le, T. P. T., Mayadunne,
R. T. A., Meijs, G. F., Moad, C. L., Moad, G., Rizzardo, E., Thang, S. H., 1998. Living Free-Radical Polymerization by Reversible Addition-Fragmentation Chain Transfer: The RAFT Process. Macromolecules 31, 5559-5562.
Czogalla, J., Schweda, F., Loffing, J., 2016. The Mouse Isolated Perfused Kidney Technique. Journal of Visualized Experiments 54712.
Ehling, J., Bdblokovd, J., Gremse, F., Klinkhammer, B. M., Baetke, S., Knuechel, R., Kiessling, F., Floege, J., Lammers, T., Boor, P., 2016. Quantitative Micro-Computed Tomography Imaging of Vascular Dysfunction in Progressive Kidney Diseases. JASN 27, 520-532.
Ghanavati, S., Yu, L. X., Lerch, J. P. & Sled, J. G. A perfusion procedure for imaging of the mouse cerebral vasculature by X-ray micro-CT. J. Neurosci. Methods 221, 70-77 (2014) Hlushchuk R, Zubler C, Barre S, Correa Shokiche C, Schaad L, Rothlisberger R, Wnuk M,
Daniel C, Khoma O, Tschanz S A, Reyes M, and Djonov V, 2018. Cutting-edge microangio-CT: new dimensions in vascular imaging and kidney morphometry. Am J Physiol Renal Physiol 314: F493-F499.
Jian, Z., Baier, M. C. & Mecking, S. Suppression of Chain Transfer in Catalytic Acrylate Polymerization via Rapid and Selective Secondary Insertion. J. Am. Chem. Soc. 137, 2836-2839 (2015)
W. Kuo, G. Schulz, B. Müller, and V. Kurtcuoglu, “Evaluation of metal nanoparticle- and plastic resin-based X-ray contrast agents for kidney capillary imaging,” in Developments in X-Ray Tomography XII, San Diego, United States, 2019, p. 23
Lai, J. T., Filla, D., Shea, R., 2002. Functional Polymers from Novel Carboxyl-Terminated Trithiocarbonates as Highly Efficient RAFT Agents. Macromolecules 35, 6754-6756.
Lantuejoul, C., Beucher, S., 1981. On the use of the geodesic metric in image analysis. Journal of Microscopy 121, 39-49.
Legland, D., Arganda-Carreras, I., Andrey, P., 2016. MorphoLibJ: integrated library and plugins for mathematical morphology with ImageJ. Bioinformatics btw413.
Migneault, I., Dartiguenave, C., Bertrand, M. J. & Waldron, K. C. Glutaraldehyde: behavior in aqueous solution, reaction with proteins, and application to enzyme crosslinking. Biotechniques 37, 790-802 (2004).
Ngo, J. P., Kar, S., Kett, M. M., Gardiner, B. S., Pearson, J. T., Smith, D. W., Ludbrook, J., Bertram, J. F., Evans, R. G., 2014. Vascular geometry and oxygen diffusion in the vicinity of artery-vein pairs in the kidney. American Journal of Physiology-Renal Physiology 307, F1111-F1122.
Ollion, J., Cochennec, J., Loll, F., Escude, C., Boudier, T., 2013. TANGO: a generic tool for high-throughput 3D image analysis for studying nuclear organization. Bioinformatics 29, 1840-1841.
Prommer, H.-U., Maurer, J., von Websky, K., Freise, C., Sommer, K., Nasser, H., Samapati, R., Reglin, B., Guimaraes, P., Pries, A. R., Querfeld, U., 2018. Chronic kidney disease induces a systemic microangiopathy, tissue hypoxia and dysfunctional angiogenesis. Sci Rep 8, 5317.
Schindelin, J., Arganda-Carreras, I., Frise, E., Kaynig, V., Longair, M., Pietzsch, T., Preibisch, S., Rueden, C., Saalfeld, S., Schmid, B., Tinevez, J.-Y., White, D. J., Hartenstein, V., Eliceiri, K., Tomancak, P., Cardona, A., 2012. Fiji: an open-source platform for biological-image analysis. Nat Methods 9, 676-682.
Schneider, C. A., Rasband, W. S., Eliceiri, K. W., 2012. NIH Image to ImageJ: 25 years of image analysis. Nat Methods 9, 671-675.
Vasquez S X, Gao F, Su F, Grijalva V, Pope J, Martin B, Stinstra J, Masner M, Shah N, Weinstein D M, Farias-Eisner R, and Reddy S T, 2011. Optimization of microCT imaging and blood vessel diameter quantitation of preclinical specimen vasculature with radiopaque polymer injection medium. PLoS One 6: e19099.
Y. K. Chong, G. Moad, E. Rizzardo and S. H. Thang, “Thiocarbonylthio End Group Removal from RAFT-Synthesized Polymers by Radical-Induced Reduction” Macromolecules, 2007, 40, 4446;
A. O. Moughton, K. Stubenrauch and R. K. O'Reilly, “Hollow nanostructures from self-assembled supramolecular metallo-triblock copolymers” Soft Matter, 2009, 5, 2361.
C. P. Jesson, C. M. Pearce, H. Simon, A. Werner, V. J. Cunningham, J. R. Lovett, M. J. Smallridge, N. J. Warren and S. P. Armes, “H₂O₂ Enables Convenient Removal of RAFT End-Groups from Block Copolymer Nano-Objects Prepared via Polymerization-Induced Self-Assembly in Water” Macromolecules, 2017, 50, 182.

1. A monomer M, or a salt thereof, comprising a backbone with 2 to 6 elements B, wherein
each B independently from any other B is selected from —CH2-, —NH—, —C(═O)—, —CH(R)— and —NR—, wherein
B may optionally be substituted by a Ci-4-alkyl, in particular by ethyl or methyl, more particularly by methyl, and wherein
at least one of the moieties —CH(R)— and —NR— is present in the backbone,
R is independently selected from -E-H, -L-(NH₂)m, and a moiety of formula 1, 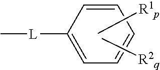
(1), wherein
E is a moiety comprising one or more moieties, particularly 1 to 3 moieties, independently selected from —C(═O)—, —NH—C(=0)-, —O—, -Ci-4-alkyl-,
L is a linker comprising one or more moieties, particularly 1 to 3 moieties, independently selected from —C(=0)-, —C(=0)-NH—, —NH—C(=0)-, -0-, —C1-4-alkyl-, wherein L may optionally be substituted by -E-H,
R¹ is —I,
R² is -E-H or -L-(NH₂)m,
p is independently selected from 0, 1, 2 or 3
q is independently selected from 0, 1, 2, 3 or 4, particularly 0, 1 or 2, wherein the sum of p and q in formula 1 is £5,
m is independently selected from 1 or 2, wherein the sum of all m in the monomer is ³ 1, particularly ³ 2, and
the sum of all p in the monomer is ³ 1, particularly the sum is 2 or 3, more particularly the sum is 3.

2. The monomer M according to claim 1, wherein each B independently from any other B is selected from —CH₂—, —NH—, —C(=0)- and —CH(R)—.

3. The monomer M according to claim 1, wherein the backbone is
a peptide backbone —C(=0)-CH(R′)—NH—C(=0)-CH(R″)—NH— or —C(=0)-CH(R′)—NH—, or
an aliphatic backbone —CH₂—CH(R′)—CH₂—CH(R″)—, —CH₂—CH(R′)—, —CH₂—C(CH₃)(R′)—CH₂—C(CH₃)(R″)— or —CH₂—C(CH₃)(R′)—, particularly —CH₂—CH(R′)—CH₂—CH(R″)— or —CH₂—CH(R′)—, more particularly —CH₂—CH(R′)—, wherein
R′ and R″ consist of moieties that are selected from moieties as defined for R,
wherein R′ and R″ differ from each other, particularly one of R′ and R″ is a moiety of formula 1 and the other one is -E-H or -L-(NH₂)ₘ, more particularly R′ is a moiety of formula 1 and R″ is -E-H or -L-(NH₂)ₘ.

4. The monomer M according to claim 1, wherein -E-H is independently selected from —OH, -Ci-4-alkyl-OH, —C(=0)-0H, -Ci-4-alkyl-C(=0)-0H, —O-Ci-4-alkyl and -Ci-4-alkyl-O-Ci-4-alkyl, particularly from —OH and —C(=0)-0H.

5. The monomer M according to claim 1, wherein R is independently selected from -L-(NH₂)ₘ and a moiety of formula 1.

6. The monomer M according to claim 1, wherein in case of R being -L-(NH₂)ₘ, -L-(NH₂)ₘ is independently selected from -Ci-4-alkyl-NH₂, -Ci-4-alkyl-C(=0)-NH₂, —C(=0)-NH₂, —C(=0)-NH—Ci-4-alkyl-NH₂, —NH—C(=0)-C₁₋₄-alkyl-NH₂ and -0-Ci-4-alkyl-NH₂, particularly from —C(=0)-NH₂ or -Ci-4-alkyl-NH₂.

7. The monomer M according to claim 1, wherein in case of R² being -L-(NH₂)ₘ, -L-(NH₂)ₘ is independently selected from -Ci-4-alkyl-NH₂, -Ci-4-alkyl-C(=0)-NH₂, —C(=0)-NH₂, —C(=0)-NH—Ci-4-alkyl-NH₂, —NH—C(=0)-C₁₋₄-alkyl-NH₂ and -0-Ci-4-alkyl-NH₂, particularly from —C(=0)-NH—Ci-₂-alkyl-NH₂.

8. A polymer P comprising monomers M according to claim 1, particularly 70 to 600, particularly 100 to 300, more particularly 120 to 170 monomers M.

9. The polymer according to claim 8, wherein the polymer is a compound of formula 2,
2a or 3, particularly of formula 2 and 2a, more particularly of formula 2,
X-[M]ₙ-Y (2), Z-[M]ₙ-R^{s} (2a), R^{N}-[M]ₙ-R^{c} (3), wherein
X and Y are independently from each other selected from RA, FT and Rl,
Z is selected from FT and Rl, wherein
Rl is a moiety derived from a radical initiator, particularly from a radical initiator selected from a peroxide, a perester or an azo initiator, more particularly from AIBN, 1,T-azobis (cyclohexanecarbonitrile), 4,4′-azobis(4-cyanopentanoic acid), 4,4′-azobis(4-cyanopentan-1-ol), 2,2′-azobis(methyl isobutyrate), 2,2′-azobis(2-cyano-2-butane), 2-(t-butylazo)-2-cyanopropane, 2,2′-azobis(N,N′-dimethylencisobutyramine), 2,2′-azobis[2-methyl-(N)-(1,1)-bis(hydroxymethyl)-2-hydroxyethyl]propionamide, 2,2′-azobis[2-methyl-N-hydroxyethyl)]propionamide, 2,2′-azobis(2,2,4-trimethylpentane), 2,2′-azobis(2-methylpropane), t-butylperoxy isobutyrate, dibenzoyl peroxide, potassium peroxydisulfate, ammonium peroxydisulfate, di-tert-butyl peroxide, di-t-butyl hyponitrite, dicumyl hyponitrite,
RA is a RAFT (reversible addition fragmentation chain transfer) agent without the homolytic leaving group,
M is a monomer according to claim 1 or a salt thereof,
n is 70 to 600, particularly 100 to 300, more particularly 120 to 170,
FT is the homolytic leaving group of a RAFT agent or the homolytic leaving group of a RAFT agent modified by -E-H or -L-(NH₂)m, wherein -E-H and -L-(NH2)ₘ are defined as described above,
R^{s} is H or OH
R^{N} is —IMH2,
R^{c} is —COOH or —CONH₂.

10. The polymer according to claim 9, wherein
Rl is a moiety of formula 5 or 6, 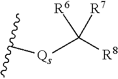
(5), —O—SO2-O-T⁺ (6), wherein
R⁶ is selected from -Ci-₆-alkyl, —H,
R⁷ is selected from -Ci-₆-alkyl, -phenyl, -Ci-₆-alkyl-OH, -Ci-₆-alkyl-COOH, —COOH, —C(=0)-0-Ci-₄-alkyl, —C(=0)-NH—R⁹ with R⁹ being —C₁₋₆-alkyl-(OH)ᵣ with r being 0, 1, 2 or 3,
R⁸ is -Ci-₆-alkyl, —H, —CN, or
R⁶ and R⁷ form a C3-8-cycloalkyl, particularly a C5-6-cycloalkyl, and R⁸ is -Ci-₆-alkyl, —H, —CN,
Q is -0-, -0-C(=0)- or —C(=0)-0- with s being 0 or 1,
T⁺ is a monovalent cation, particularly Na⁺, K⁺, Nh, H⁺, and/or
RA is —S—C(═S)—Z with Z being selected from phenyl and —S—C6-2o-alkyl, particularly phenyl and —S-Cio-16-alkyl, more particularly —S-Cio-16-alkyl, and/or
FT is a moiety of formula 4, 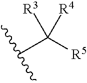
(4), wherein
R³ is selected from —H and -Ci-4-alkyl, particularly —H and -Ci-2-alkyl,
R⁴ is selected from —H, -Ci-4-alkyl, -Ci-4-alkyl-COOH, -Ci-4-alkyl-C(=0)-R⁶ with R⁶ being -E-H or -L-(NH₂)ₘ, particularly —H, -Ci-2-alkyl, -Ci-2-alkyl-COOH, -Ci-2-alkyl-C(=0)-R⁵ with R⁵ being -E-H or -L-(NH₂)ₘ,
R⁵ is selected from —CN and —COOH, particularly R^{S} is —COOH.

11. A pre-crosslinked polymer comprising two or more interconnected polymers P according to claim 8.

12. The pre-crosslinked polymer according to claim 11, wherein the polymers are interconnected via imine bonds, which are formed by a reaction of amine moieties of the polymers and a dialdehyde, particularly H—C(=0)-Ci-₈-alkyl-C(=0)-H, more particularly H—C(=0)-C₃₋₈-alkyl-C(=0)-H, or a trialdehyde, particularly benzene-1, 3,5-trialdehyde, or the polymers are interconnected via a methylene bridge derived from formaldehyde.

13. The pre-crosslinked polymer according to claim 11, wherein the molecular mass of the pre-crosslinked polymer is ³ 65 kDa, particularly ³ 100 kDa.

14. An intermediate M′ of formula 5, D-CH(R) (5), wherein D is H2C═ and R is a moiety of formula 1, 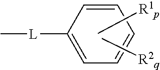
(1) as defined above.

15. A method for ex vivo imaging, particularly vascular and renal tubular imaging, comprising the steps of
providing a contrast agent solution comprising the pre-crosslinked polymer according to claim 12 and a crosslinking solution comprising a crosslinking agent, particularly a crosslinking agent selected from formaldehyde, a dialdehyde or a trialdehyde,
perfusing a vessel using the contrast agent solution, particularly perfusing the vasculature of a tissue, an organ or a whole animal,
adding the crosslinking solution yielding a crosslinked polymer,
detecting the crosslinked polymer using X-ray.
